(19) United States
(12) Patent Application Publication
(10) Pub. No.: US 2024/0024491 A1
(43) Pub. Date: Jan. 25, 2024

(54) MANNOSYLATED AMINE DEXTRAN DRUG DELIVERY VEHICLES WITH DEGRADABLE DISULFIDE/CARBONATE LINKERS TARGETING PAYLOADS TO CD206 EXPRESSING CELLS

(71) Applicant: Navidea Biopharmaceuticals, Inc., Dublin, OH (US)

(72) Inventors: Jeffrey Scott Arnold, Andover, MA (US); David A. Ralph, Columbus, OH (US)

(21) Appl. No.: 18/351,905

(22) Filed: Jul. 13, 2023

Related U.S. Application Data
(60) Provisional application No. 63/388,777, filed on Jul. 13, 2022.

(51) Int. Cl. A61K 47/55; A61K 47/61; A61K 47/54; A61P 35/00; A61K 39/395

(52) U.S. Cl. CPC A61K 47/55; A61K 47/61; A61K 47/542; A61P 35/00; A61K 39/3955

(57) ABSTRACT

Provided are compounds, compositions, and methods of repolarizing a tumor associated macrophage (TAM), reducing macrophage-mediated inflammation, and treating a disease. A compound or pharmaceutical composition may be administered to a subject in need thereof, where the compound comprises a polymeric carbohydrate backbone, one or more mannose-binding C-type lectin receptor targeting moieties, and a therapeutic agent comprises one or more reactive hydroxyl groups and coupled to the polymeric carbohydrate backbone via a degradable linker. The degradable linker may comprise one or more carbonate and/or disulfide moieties. The disease to be treated may include cancer, an autoimmune disease, an inflammatory disorder, Non-Alcoholic Steatohepatitis (NASH), acute respiratory distress syndrome (ARDS), sepsis, coronavirus infection, influenza infection, cytokine storms, and other macrophage involved diseases.

### CROSS-REFERENCE TO RELATED APPLICATION(S)

This application claims the benefit of U.S. Provisional Application Ser. No. 63/388,777, filed on Jul. 13, 2022 and entitled “Mannosylated Amine Dextran Drug Delivery Vehicles with Degradable Disulfide/Carbonate Linkers Targeting Payloads to CD206 Expressing Cells,” the entire contents of which are incorporated herein by reference.

### TECHNICAL FIELD

The disclosure relates to therapeutic constructs, methods of making, and methods of use thereof for the targeted delivery of therapeutic payloads, more specifically, therapeutic payloads having a reactive hydroxyl group, and releasing the therapeutic payload when internalized into a mannose-binding C-type lectin receptor-expressing cell and/or shifting the phenotype toward a proinflammatory state.

### BACKGROUND

Various C-type lectin receptors may be expressed on macrophages, dendritic cells, and mesangial cells, with the mannose receptor (CD206) being one example of a C-type lectin receptor. The CD206 receptor typically binds to molecules that display multiple terminal mannose moieties, and once CD206 binds to a ligand, the receptor/ligand complex is internalized via receptor mediated endocytosis to endosomes that become naturally acidified to a pH of between about 4 to 5. At this lower pH level, CD206 releases its ligand and recycles to the cell surface. Besides pH, various enzymes are released into endosomes that can act on endosomal contents.

Macrophages are a common cell type in all tissues of the body and are important components of innate immunity. Macrophages also contribute significantly to the maintenance of tissue homeostasis and wound repair. An important feature of macrophages includes the ability to adopt many phenotypic states in response to various stimuli from their local environment. Furthermore, macrophages can change their phenotypic state if the stimuli from their local environment changes. When macrophages change their phenotype in response to environmental stimuli, they are referred to as activated macrophages. Activated macrophages alter their expression of hundreds of genes. Furthermore, the genes whose expressions are changed in response to one set of environmental stimuli can be significantly different from the genes whose expression is altered in response to a different set of stimuli. One of the genes that frequently undergoes increased expression upon macrophage activation is CD206. Investigations seeking to understand the gene expression changes in macrophages responding to different stimuli in various in vivo and in vitro situations have been the topic of thousands of scientific publications over the last few decades.

Extensive literature on macrophage activation have found that there are a vast number of activated phenotypes and expression of only one or a few genes cannot accurately identify any one particular phenotype. However, these activated phenotypes can be characterized for their overall immune status and can be placed on a continuum, with highly pro-inflammatory phenotypes at one end of the continuum and immunosuppressive and wound healing phenotypes at the other end. Traditionally, as referenced by historic macrophage phenotype literature, activated macrophages were divided into two phenotypes: (1) classically activated, called M1, which is highly proinflammatory, and (2) alternatively activated, called M2, which is immunosuppressive and promotes wound healing. It is now understood that a strictly dichotomous classification of activated macrophage phenotypes is overly simplistic and does not represent the true plasticity of macrophage responses to stimuli from their microenvironments; however, the concept that activated macrophages can influence a local immune response by being either proinflammatory (M1-like) or immunosuppressive (M2-like) continues to have utility when describing the role of macrophages in various pathological states.

In living humans and animals, activated macrophages have been observed that have a mixed activated phenotype with features of both M1-like and M2-like phenotypic states. Examples of stimuli that can induce an M1-like phenotype in macrophages include tumor necrosis factor (TNF), interferon gamma (INFy), and toll-like receptor (TLR) agonists such as lipopolysaccharide (LPS). Examples of stimuli that can induce an M2-like phenotype in macrophages include interleukin 4 (IL4), interleukin 13 (IL13), tumor growth factor beta (TGF8), and glucocorticoids, such as dexamethasone. There are many other hormones, cytokines, chemokines, and environmental factors that can also affect macrophage phenotypes.

Tumor associated macrophages (TAMs) are abundant in tumors and highly significant contributors to the maladaptive immune response associated with cancer and other conditions. TAMs are the most numerous immune cells that infiltrate tumors and can comprise from about 5% to >30% of all cells in a tumor. While both M1-like and M2-like TAMs are known, the large majority of TAMs residing in or near established tumors are immunosuppressive, i.e., M2-like activated macrophages.

Accordingly, there remains a need for compositions and methods that induce the phenotypic change of the M2-like TAMs to M1-like TAMs in order to treat cancer with greater efficacy and lower toxicity.

Other objects, advantages and features of the present disclosure will become apparent from the following specification taken in conjunction with the accompanying figures.

### BRIEF SUMMARY

In Example 1, a compound comprises a polymeric carbohydrate backbone, one or more mannose-binding C-type lectin receptor targeting moieties; and a therapeutic agent comprising one or more reactive hydroxyl groups, and coupled to the polymeric carbohydrate backbone via a degradable linker.

Example 2 relates to the compound according to Example 1, wherein the compound comprises a subunit as shown in Formula (I):

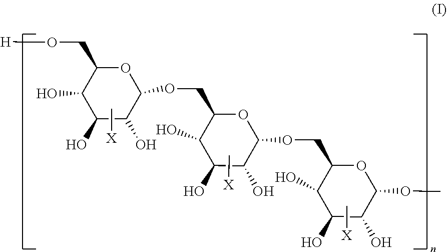

wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

Example 3 relates to the compound according to Example 1 or 2, wherein at least one X is L₁-A-Z, wherein at least one X is L₂-R, and wherein R comprises the mannose-binding C-type lectin receptor targeting moiety.

Example 4 relates to the compound according to any one of Examples 1-3, wherein the polymeric carbohydrate backbone has a molecular weight between about 1 kD to about 50 kD.

Example 5 relates to the compound according to any one of Examples 1-4, wherein the mannose-binding C-type lectin receptor targeting moiety comprises a mannosyl coupling reagent, mannose, high-mannose glycans or mannose oligosaccharides, fucose, n-acetylglucosamine, peptides, galactose, or a combination thereof.

Example 6 relates to the compound according to any one of Examples 2-5, wherein at least one L₁ and/or at least one L₂ comprises —(CH₂)ₚS(CH2) q—NH—, wherein p and q are integers from 0 to 5.

Example 7 relates to the compound according to any one of Examples 2-6, wherein the therapeutic agent is conjugated to the degradable linker prior to being coupled to the polymeric carbohydrate backbone.

Example 8 relates to the compound according to any one of Examples 1-7, wherein the degradable linker comprises one or more carbonate and/or disulfide moieties.

Example 9 relates to the compound according to any one of Examples 2-8, wherein the degradable linker has the following formula below prior to being conjugated to the therapeutic agent and the polymeric carbohydrate backbone:

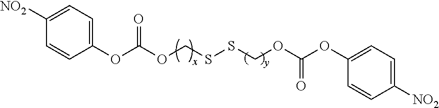

wherein x is an integer between 1-5, and wherein y is an integer between 1-5.

Example 10 relates to the compound according to any one of Examples 2-8, wherein the degradable linker has the following formula below prior to being conjugated to the therapeutic agent and the polymeric carbohydrate backbone:

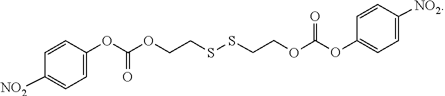 [SMILES: O=C(OCCSSCCOC(=O)Oc1ccc([N+](=O)[O-])cc1)Oc1ccc([N+](=O)[O-])cc1]

Example 11 relates to the compound according to any one of Examples 2-10, wherein A has the following formula:

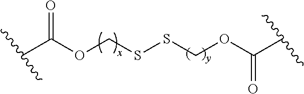

wherein x is an integer between 1-5, and wherein y is an integer between 1-5.

Example 12 relates to the compound according to any one of Examples 2-11, wherein A has the following formula:

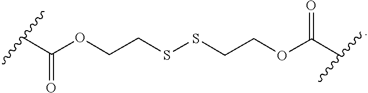 [SMILES: *C(=O)OCCSSCCOC(*)=O]

Example 13 relates to the compound according to any one of Examples 1-12, wherein the therapeutic agent comprises a corticosteroid, a cortisol, a glucocorticoid-receptor ligand, a chemotherapeutic agent, a toll-like receptor agonist or antagonist, or a combination thereof.

Example 14 relates to the compound according to any one of Examples 1-13, wherein the therapeutic agent comprises dexamethasone or paclitaxel.

In Example 15, a pharmaceutical composition comprises the compound according to any one of Examples 1-14, and a pharmaceutically effective carrier.

Example 16 relates to the composition according to Example 15, wherein the compound comprises a subunit as shown in Formula (I):

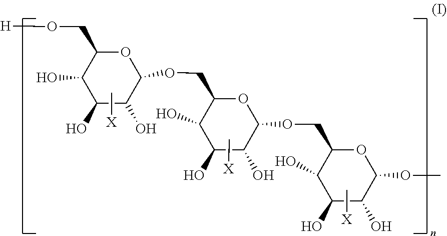

wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

In Example 17, a compound comprises a therapeutic agent comprising a reactive hydroxyl group;
a degradable linker comprising one or more carbonate and/or disulfide moieties; and a second compound comprising a primary amine, wherein the degradable linker is coupled to the reactive hydroxyl group of the therapeutic agent and coupled to the primary amine of the second compound.

Example 18 relates to the compound according to Example 17, wherein compound has the following formula (II):

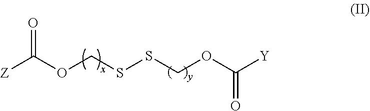

wherein
Z is the therapeutic agent comprising a reactive hydroxyl group;
Y is the second compound comprising a primary amine;
x is an integer between 1-5; and
y is an integer between 1-5.

Example 19 relates to the compound according to Example 17 or 18, wherein the therapeutic agent comprises a corticosteroid, cortisol, a glucocorticoid-receptor ligand, a chemotherapeutic agent, a toll-like receptor agonist or antagonist, or a combination thereof.

Example 20 relates to the compound according to any one of Examples 17-19, wherein the therapeutic agent comprises dexamethasone or paclitaxel.

In Example 21, a method of making the compound according to any one of Examples 1-14 comprises (a) synthesizing a polymeric carbohydrate backbone having one or more amine terminated leashes attached thereto; (b) synthesizing a degradable linker comprising one or more carbonate and/or disulfide moieties; (c) reacting the degradable linker with a reactive hydroxyl group of the therapeutic agent to form a therapeutic-linker compound; and (d) reacting the therapeutic-linker compound with the one of the one or more amine terminated leashes on the polymeric carbohydrate backbone.

Example 22 relates to the method according to Example 21, wherein step (a) may occur before step (b), (c), or (d), or may occur after step (b) or (c).

Example 23 relates to the method according to Example 21 or 22, wherein the therapeutic agent is conjugated to the degradable linker prior to being coupled to the polymeric carbohydrate backbone.

Example 24 relates to the method according to any one of Examples 21-23, wherein at least one X is L₁-A-Z, wherein at least one X is L₂-R, and wherein R comprises the mannose-binding C-type lectin receptor targeting moiety.

Example 25 relates to the method according to any one of Examples 21-24, wherein the polymeric carbohydrate backbone has a molecular weight between about 1 kD to about 50 kD.

Example 26 relates to the method according to any one of Examples 21-25, wherein the mannose-binding C-type lectin receptor targeting moiety comprises a mannosyl coupling reagent, mannose, high-mannose glycans or mannose oligosaccharides, fucose, n-acetylglucosamine, peptides, galactose, or a combination thereof.

Example 27 relates to the method according to any one of Examples 21-26, wherein at least one L₁ and/or at least one L₂ comprises —(CH₂)ₚS(CH2) q—NH—, wherein p and q are integers from 0 to 5.

Example 28 relates to the method according to any one of Examples 21-27, wherein the degradable linker has the following formula below prior to being conjugated to the therapeutic agent and the polymeric carbohydrate backbone:

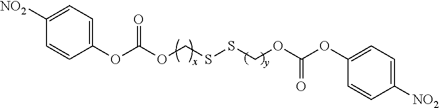

wherein x is an integer between 1-5, and wherein y is an integer between 1-5.

Example 29 relates to the method according to any one of Examples 21-28, wherein the degradable linker has the following formula below prior to being conjugated to the therapeutic agent and the polymeric carbohydrate backbone:

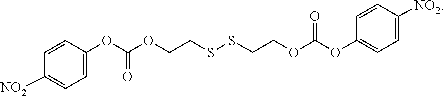 [SMILES: O=C(OCCSSCCOC(=O)Oc1ccc([N+](=O)[O-])cc1)Oc1ccc([N+](=O)[O-])cc1]

Example 30 relates to the method according to any one of Examples 21-29, wherein A has the following formula:

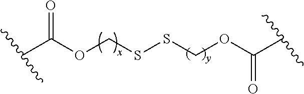

wherein x is an integer between 1-5, and wherein y is an integer between 1-5.

Example 31 relates to the method according to any one of Examples 21-30, wherein A has the following formula:

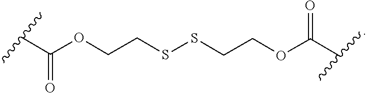 [SMILES: *C(=O)OCCSSCCOC(*)=O]

Example 32 relates to the method according to any one of Examples 21-31, wherein the therapeutic agent comprises a corticosteroid, cortisol, a glucocorticoid-receptor ligand, a chemotherapeutic agent, a toll-like receptor agonist or antagonist, or a combination thereof.

Example 33 relates to the method according to any one of Examples 21-32, wherein the therapeutic agent comprises dexamethasone, paclitaxel, or a combination thereof.

In Example 34, a method of repolarizing a tumor associated macrophage (TAM) from an immunosuppressive (M2-like) phenotype to a proinflammatory (M1-like) phenotype comprises administering to a subject in need thereof an effective dose of a compound comprising a polymeric carbohydrate backbone, one or more mannose-binding C-type lectin receptor targeting moieties, and a therapeutic agent comprising one or more reactive hydroxyl groups, and coupled to the polymeric carbohydrate backbone via a degradable linker.

Example 35 relates to the method according to Example 24, wherein the compound comprises a subunit as shown in Formula (I):

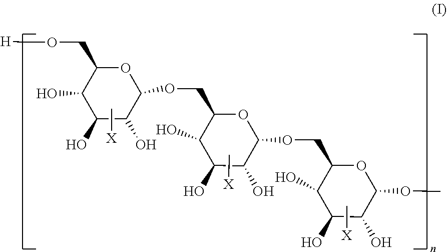

wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

Example 36 relates to the method according to Example 34 or 35, wherein the compound is administered in conjunction with at least one other therapy or treatment and, wherein the at least one other treatment or therapy is a chemotherapy, radiation therapy, or immunotherapy.

Example 37 relates to the method according to any one of Examples 34-36, wherein the therapeutic agent comprises paclitaxel.

Example 38 relates to the method according to any one of Examples 34-37, wherein the subject in need thereof is suffering from cancer.

Example 39 relates to the method according to any one of Examples 34-38, wherein the therapeutic agent is released from the polymeric carbohydrate backbone in the presence of a reducing agent.

Example 40 relates to the method according to any one of Examples 34-39, wherein the method does not repress the anti-tumor activity of lymphocytes.

In Example 41, a method of reducing macrophage-mediated inflammation comprises administering to a subject in need thereof an effective dose of a compound comprising a polymeric carbohydrate backbone, one or more mannose-binding C-type lectin receptor targeting moieties, and a therapeutic agent comprising one or more reactive hydroxyl groups, and coupled to the polymeric carbohydrate backbone via a degradable linker.

Example 42 relates to the method according to Example 41, wherein the compound comprises a subunit as shown in Formula (I):

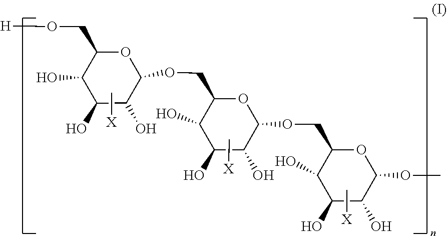

wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

Example 43 relates to the method according to Example 41 or 42, wherein the compound is administered in conjunction with at least one other therapy or treatment.

Example 44 relates to the method according to any one of Examples 41-43, wherein the therapeutic agent comprises dexamethasone.

Example 45 relates to the method according to any one of Examples 41-44, wherein the subject in need thereof is suffering from Non-Alcoholic Steatohepatitis (NASH), acute respiratory distress syndrome (ARDS), sepsis, coronavirus infection, influenza infection, cytokine storms, other macrophage involved diseases, or a combination thereof.

Example 46 relates to the method according to any one of Examples 41-45, wherein the therapeutic agent is released from the polymeric carbohydrate backbone in the presence of a reducing agent.

Example 47 relates to the method according to any one of Examples 41-46, wherein the method does not repress the anti-tumor activity of lymphocytes.

In Example 48, a method of treating a disease comprises administering to a subject in need thereof an effective amount of a compound according to any one of Examples 1-14, wherein the disease is selected from the group consisting of cancer, an autoimmune disease, an inflammatory disorder, Non-Alcoholic Steatohepatitis (NASH), acute respiratory distress syndrome (ARDS), sepsis, coronavirus infection, influenza infection, cytokine storms, and other macrophage involved diseases.

Example 49 relates to the method according to Example 48, wherein the compound is administered in conjunction with at least one other therapy or treatment.

Example 50 relates to the method according to Example 49, wherein the at least one other treatment or therapy is a chemotherapy, radiation therapy, or immunotherapy.

Example 51 relates to the method according to any one of Examples 48-50, wherein the therapeutic agent comprises a corticosteroid, cortisol, a glucocorticoid-receptor ligand, a chemotherapeutic agent, a toll-like receptor agonist or antagonist, or a combination thereof.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE FIGURES

FIG. 1 shows the synthesis of various compounds as a result of a reaction between dexamethasone and a hydrazide.

FIG. 2 shows the synthesis of a dexamethasone-carbonate compound and a MAD-dexamethasone conjugate utilizing the methods of the present disclosure.

FIG. 3 shows a line graph providing the weight percent of dexamethasone released from the MAD-dexamethasone conjugate in relation to time in hours.

FIG. 4 shows a bar graph of how the various treatments with either MAD-DEX3.5 (MAD-DEX built on a 3.5 kDa dextran backbone) or free dexamethasone affected the level of expression of eight evaluated surface markers. All experiments were run in triplicate with macrophages from three human donors. The significance was determined by a Z test comparing Drug Treated v. No Drug controls.

FIG. 5 shows a bar graph of results demonstrating that AF488-MAD-DEX3.5 binds selectively to macrophages and that only macrophages bound the anti-CD206 antibody, indicating that MAD-DEX3.5 delivers dexamethasone preferentially to CD206 expressing cells.

FIG. 6 shows the synthesis of a paclitaxel-carbonate compound and a MAD-paclitaxel conjugate utilizing the methods of the present disclosure.

FIG. 7 shows a bar graph of results demonstrating that AF488-MAD-PAC3.5 binds preferentially to macrophages, indicating that MAD-PAC3.5, and similar MAD-PAC constructs built on dextran backbones of different molecular weights, will deliver paclitaxel preferentially to CD206 expressing cells.

FIG. 8 shows a bar graph of how the various treatments with either MAD-PAC3.5 (MAD-PAC built on a 3.5 kDa dextran backbone) or free paclitaxel affected the level of expression of eight evaluated surface markers. All experiments were run in triplicate with macrophages from three human donors. Within the graph, the line at 1.0 indicates no change from the no drug control. The significance was determined by a Z test comparing Drug Treated v. No Drug controls.

FIG. 9 shows a bar graph showing the results for the mean tumor volumes per treatment group observed on treatment day 14 for treatment groups for saline, free paclitaxel, MAD-Pac, CTLA4, free paclitaxel and CTLA4, and MAD-Pac and CTLA4.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular compounds, compositions, and methods, which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, and time. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. It is also contemplated that, in certain aspects, unless expressly indicated to the contrary, individual substituents can be further optionally substituted (i.e., further substituted or unsubstituted).

In defining various terms, “A₁,” “A₂,” “A₃,” “A₄,” “X₁,” “X₂,” “Y₁,” “Y₂,” etc. are used herein as generic symbols to represent various specific substituents. These symbols can be any substituent, not limited to those disclosed herein, and when they are defined to be certain substituents in one instance, they can, in another instance, be defined as some other substituents.

“R₁,” “R₂,” “R₃,” “Rₙ,” where n is an integer, as used herein can, independently, possess one or more of the groups listed above. For example, if R₁ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “polarization” is used herein to designate the phenotypic features and the functional features of the macrophages. The phenotype can be defined through the surface markers expressed by the macrophages. The functionality can be defined, for example, based on the nature and the quantity of chemokines and/or cytokines expressed, in particular, secreted by the macrophages. Indeed, the macrophages present different phenotypic and functional features depending of their state, either pro-inflammatory (M1-type) macrophage or anti-inflammatory (M2-type) macrophage. M2-type macrophages can be characterized by the expression of surface markers such as CD206, CD11b, PD-LI and CD200R and then secretion of cytokines such as CCL17. M1-type macrophages can be defined by the expression of surface markers such as CD86 and CCR7 and the secretion of cytokines such as IL-6, TNF-α and IL12p40. In the context of the present disclosure, the term “repolarize” is used herein to refer to the induction of a change in phenotype of M1 macrophages population to M1-type macrophages.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “synergistic” means that the effect achieved with the methods and combinations of this invention is greater than the sum of the effects that result from using the compounds, compositions, treatments and/or methods a pharmaceutically acceptable salt thereof, separately. Advantageously, such synergy provides greater efficacy at the same doses, and/or prevents or delays the build-up of multi-drug resistance.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an ICso of the particular compound as measured in an in vitro assay. Calculating dosages to achieve such circulating blood or scrum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

The phrase “anti-cancer composition” can include compositions that exert antineoplastic, chemotherapeutic, antiviral, antimitotic, antitumorgenic, anti-angiogenic, anti-metastatic and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids. The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

The present disclosure describes compounds, compositions, methods with utility for altering or transitioning the phenotype of activated macrophages from being an immunosuppressive phenotype to a proinflammatory phenotype (referred to herein as “repolarizing”), and methods with utility for increasing target specificity of the compounds or compositions in a subject. The ability to alter or transition the phenotype of activated macrophages from immunosuppressive to proinflammatory constitutes a therapeutic modality for cancer, various infectious diseases and other medical conditions. The present disclosure further describes a drug delivery vehicle and methods of use that enables the targeted delivery of therapeutic agents to TAMs, including with the intent to repolarize the TAMs. TAM-targeted delivery provides higher mass doses of the therapeutic agents to TAMs— increasing phenotype altering effects—while limiting potentially toxic exposure to off target cells and tissues. Through the use of the disclosed compounds, compositions and methods, M2-like (immunosuppressive) activated macrophages can be induced to switch their phenotype to a M1-like (proinflammatory) activated phenotype. In further embodiments, the disclosure provides for methods of reducing macrophage-mediated inflammation in a subject.

### Compounds and Compositions

In certain aspects, compounds disclosed herein employ a carrier construct comprising a polymeric (e.g. carbohydrate) backbone having conjugated thereto mannose-binding C-lectin type receptor targeting moieties (e.g. mannose) to deliver one or more active therapeutic agents. Examples of such polymeric carbohydrate constructs include mannosylated amine dextrans (MADs), which comprise a dextran backbone having mannose molecules conjugated to glucose residues of the backbone and having an active pharmaceutical ingredient conjugated to glucose residues of the backbone. Tilmanocept is a specific example of a MAD. A tilmanocept derivative that is tilmanocept without DTPA conjugated thereto is a further example of a MAD.

MADs are synthetic molecules purposefully designed to be high affinity ligands for mannose-binding C-lectin type receptors, such as, for example, CD206. MADs have been described in U.S. Pat. No. 6,409,990, which is hereby incorporated by reference in its entirety. Thus, the backbone comprises a plurality of glucose moieties (i.e., residues or subunits) primarily linked by a-1,6 glycosidic bonds. Other linkages such as a-1,4 and/or a-1,3 bonds may also be present.

Some embodiments may comprise a backbone that is not a dextran backbone. Some embodiments may have a monosaccharide-based backbone that does not comprise dextran. The backbone of a carbohydrate-based carrier molecules described herein can comprise a glycan other than dextran, wherein the glycan comprises a plurality of monosaccharide residues (i.e., sugar residues or modified sugar residues). In certain embodiments, the glycan backbone has sufficient monosaccharide residues, as well as optional groups such as one or more amino acids, polypeptides and/or lipids, to provide a MW of about 1 to about 50 kDa. As would be appreciated by the skilled artisan when considering the disclosure contained herein, when referring to a “dextran” backbone, other monosaccharide residues may be considered to be substituted in compounds described herein. Additional descriptions of carbohydrate-backbone-based carrier molecules used for targeting CD206 are described in PCT application No. US/2017/055211, which is herein incorporated by reference in its entirety.

In some embodiments, not every backbone moiety is substituted. In some embodiments, one or more amine terminated leashes are attached to the backbone. In further embodiments, mannose-binding C-type lectin receptor targeting moieties are attached to the one or more amine terminated leashes. In certain embodiments, the mannose-binding C-type lectin targeting moieties are attached to between about 15% and about 70%, between about 17% and about 65%, or about 20% and about 60% of the glucose residues via the amine terminated leashes. In further embodiments, the mannose-binding C-type lectin targeting moieties are attached to up to about 60%, up to about 70%, up to about 80%, up to about 90%, or up to about 100% of the glucose residues via the amine terminated leashes. In certain aspects, the percentages may vary depending on the size of the dextran backbone. In even further embodiments, one or more therapeutic agents are attached to the glucose residues via a degradable linker comprising one or more carbonate and/or disulfide moieties. The degradable linker is further conjugated to the amine terminated leashes, which is linked to the backbone described in greater detail herein. In certain embodiments, the therapeutic agents are attached to between about 1% and about 30%, between about 2% and about 25%, or about 5% and about 20% of the glucose residues via the amine terminated leash and degradable linker, as described herein.

The size of the polymeric carbohydrate constructs can be varied by changing the size of the initial polymeric carbohydrate backbone upon which the construct is assembled. In some embodiments, the polymeric carbohydrate-based moiety is between about 50-100 kDa. The polymeric carbohydrate-based moiety may be at least about 50 kDa, at least about 60 kDa, at least about 70 kDa, at least about 80 kDa, or at least about 90 kDa. The polymeric carbohydrate-based moiety may be less than about 100 kDa, less than about 90 kDa, less than about 80 kDa, less than about 70 kDa, or less than about 60 kDa. Alternatively, in some embodiments, the polymeric carbohydrate backbone has a MW of between about 1 kDa and about 50 kDa, while in other embodiments the polymeric carbohydrate backbone has a MW of between about 5 kDa and about 25 kDa. In still other embodiments, the polymeric carbohydrate backbone has a MW of between about 8 kDa and about 15 kDa, such as about 10 kDa. While in other embodiments the polymeric carbohydrate backbone has a MW of between about 1 kDa and about 5 kDa, such as about 3 kDa. Beneficially, the smaller sizes of the disclosed constructs enable greater tumor penetration and greater localization to tumor associated macrophages (TAMs) than is possible with other larger constructs.

One class of clinically important activated macrophages consists of TAMs as all tumors contain TAMs. TAMs are the most numerous immune cells that infiltrate tumors and can comprise 5% to >30% of all cells in a tumor. Tumors have both M1-like and M2-like TAMs; however, in established tumors, M2-like TAMs have a dominant influence on the immune status of the tumor microenvironment. CD206 is commonly highly expressed on a large portion of TAMs. Under the influence of the M2-like TAMs, the tumor immune microenvironment becomes tumor promoting and immunosuppressive, repressing the anti-tumor activity of other immune cells such as lymphocytes. The tumor promoting and immunosuppressive activities of M2-like TAMs reduce the efficacies of anti-cancer therapies and perhaps most notably of anti-cancer immunotherapies. For these reasons, TAMs are well recognized as a therapeutic target for cancer. TAM targeted cancer therapeutic strategies include: (1) killing or ablating TAMs; (2) blocking recruitment of TAMs to tumors; or (3) causing TAMs to switch their phenotypes from M2-like to M1-like. This third strategy is sometimes referred to as TAM repolarization or TAM reeducation. M1-like TAMs will attack tumor cells and stimulate other types of immune cells, such as lymphocytes, to do the same.

Mannosylated amine dextrans (MADs, see U.S. Pat. Nos. 6,409,990 and 10,806,803) are synthetic molecules purposefully designed to have high affinity ligands for CD206. The size of a MAD can be varied by changing the size of the initial dextran upon which the MAD construct is assembled. In some aspects, described within the present disclosure are polymeric carbohydrate constructs or MAD constructs synthesized on various molecular weight dextran backbones that carry either a therapeutic agent on a degradable linker that contains a carbonate and/or disulfide linkage. In some examples, dexamethasone is used as a potent anti-inflammatory drug through its activity as a corticosteroid hormone receptor agonist. In further examples, paclitaxel alters the inflammatory phenotype of macrophages from an immunosuppressive, M2-like phenotype towards a more proinflammatory, M1-like phenotype and improve the efficacy of other anti-cancer therapies. At neutral pH and oxidative conditions, the constructs retain their payloads long enough to carry them into an endosome. Once inside an acidified endosome with potentially reducing conditions and/or appropriate enzyme activity, the drug payload is released. These constructs have low toxicity to human macrophages but a remarkable ability to alter their phenotypes. In the case of the dexamethasone carrying MAD construct (MAD-DEX), MAD-DEX induced macrophages to adopt a more immunosuppressive, M2-like phenotype. In some embodiments, a therapeutic agent comprising paclitaxel, MAD-PAC, induced macrophages to adopt a highly proinflammatory, M1-like phenotype. In some aspects, the adoption of a highly proinflammatory M1-like phenotype is not fully duplicated by unbound (free) paclitaxel.

According to certain embodiments, and as further described throughout the disclosure, one or more mannose-binding C-type lectin receptor targeting moieties and one or more therapeutic agents are each independently attached to the polymeric carbohydrate-based backbone by way of a leash. As described in greater detail below, one or more additional moieties may be present between the leash and the mannose-binding C-type lectin receptor targeting moieties or the therapeutic agent. In further embodiments, the leash is not attached to a mannose-binding C-type lectin receptor targeting moiety or a therapeutic agent, and instead, is provided as a standalone leash attached to the polymeric carbohydrate-based backbone. The leash may be attached to from about 50% to about 100% of the backbone moieties, or from about 70% to about 90% of the backbone moieties. The leashes may be the same or different. In some embodiments, the leash is an amine terminated leash. In some embodiments, the leashes may comprise the formula —(CH₂)ₚS(CH2) q—NH—, wherein p and q are integers from 0 to 5. In further embodiments, the leash comprises the formula —(CH₂)₃S(CH2)₂NH—. In embodiments where the leash is not attached to a mannose-binding C-type lectin receptor targeting moiety or a therapeutic agent, the leash may comprise the formula —(CH₂)ₚS(CH2)_{q}—NH2, wherein p and q are integers from 0 to 5.

In some embodiments, the leash may be a chain of from about 1 to about 20 member atoms selected from carbon, oxygen, sulfur, nitrogen and phosphorus. The leash may be a straight chain or branched. The leash may also be substituted with one or more substituents including, but not limited to, halo groups, perfluoroalkyl groups, perfluoroalkoxy groups, alkyl groups, such C1-4 alkyl, alkenyl groups, such as C₁₋₄ alkenyl, alkynyl groups, such as C₁₋₄ alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═0)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH2; ═N—H; =N-alkyl; —SH; —S-alkyl; —NH—C(0)—; —NH—C(═N)— and the like. As would be apparent to one skilled in the art, other suitable leashes are possible.

According to certain embodiments, the polymeric carbohydrate constructs disclosed herein contain at least one targeting moiety. In some embodiments, the targeting moiety may be a mannose-binding C-type lectin receptor targeting moiety. In further embodiments, the polymeric carbohydrate constructs disclosed herein contain at least one targeting ligand. CD206 is a C-type lectin receptor expressed on macrophages, dendritic cells, and mesangial cells. CD206 binds to molecules that display multiple terminal mannose moieties. Without being limited to any particular mechanism or theory, it is contemplated that once CD206 binds to a ligand, the receptor/ligand complex is internalized by receptor mediated endocytosis to endosomes that become naturally acidified to a pH of about 4-5. At this lower pH, CD206 releases its ligand and recycles to the cell surface. In some aspects, the inclusion of the mannose-binding C-type lectin receptor targeting moiety within the polymeric carbohydrate construct provides numerous benefits in delivering a therapeutic agent to a target, such as macrophages.

According to some embodiments, the mannose-binding C-type lectin receptor targeting moiety comprises mannose, high-mannose glycans or mannose oligosaccharides, fucose, or n-acetylglucosamine, peptides, or galactose. In further embodiments, the mannose-binding C-type lectin receptor targeting moiety is attached to the amine terminated leash via a mannosyl coupling reagent as described in U.S. Pat. No. 6,409,990, which is hereby incorporated by reference in its entirety. As such, in some embodiments, the mannose-binding C-type lectin receptor targeting moiety comprises the mannosyl coupling reagent, mannose, high-mannose glycans or mannose oligosaccharides, fucose, n-acetylglucosamine, peptides, galactose or a combination thereof. In further embodiments, the mannose-binding C-type lectin receptor targeting moiety comprises mannose. In other embodiments, the at least one targeting ligand may be sialic acid.

In some embodiments, one or more therapeutic agents is attached to the polymeric carbohydrate backbone via a degradable linker. In some embodiments, the degradable linker comprises one or more carbonate and/or disulfide moieties. The degradable linkers release the therapeutic agent payload where free thiol groups reductively cleave the disulfide moiety of the linker. In some embodiments, when a mannosylated polymeric carbohydrate construct binds to CD206, it is internalized to endosomes which become increasingly acidified over time, thereby releasing the therapeutic agent payloads intracellularly. In an aspect, the degradable linker has the following formula below prior to being conjugated to the therapeutic agent and the polymeric carbohydrate backbone:

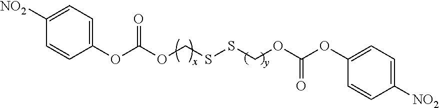

wherein x is an integer between 1-5, and wherein y is an integer between 1-5. In further aspects, the degradable linker has the following formula below prior to being conjugated to the therapeutic agent and the polymeric carbohydrate backbone:

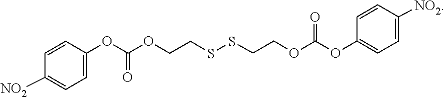 [SMILES: O=C(OCCSSCCOC(=O)Oc1ccc([N+](=O)[O-])cc1)Oc1ccc([N+](=O)[O-])cc1]

In some embodiments, the degradable linker is not a hydrazone linker.

In some embodiments, one or more therapeutic agents are attached to the polymeric carbohydrate construct. In some aspects, the one or more therapeutic agents are attached to the polymeric carbohydrate construct via the degradable linkers disclosed herein. In some aspects, the degradable linkers are further conjugated to an amine terminated leash on the polymeric carbohydrate backbone. In certain embodiments, the therapeutic agent is capable of repolarizing M2-like macrophages to M1-like macrophages when attached to the MAD backbones disclosed herein. In further embodiments, the therapeutic agent is capable of inducing T-Cell activation. In some embodiments, the therapeutic agent comprises a corticosteroid, a cortisol, a glucocorticoid-receptor ligand, a chemotherapeutic agent, a toll-like receptor agonist or antagonist, or a combination thereof. According to further embodiments, the therapeutic agent is a cytotoxic agent. In still further embodiments, the therapeutic agent is an anti-cancer agent.

In certain embodiments, the therapeutic agent comprises one or more reactive hydroxyl groups. In further embodiments, the therapeutic agent may be dexamethasone or paclitaxel. Dexamethasone is a potent anti-inflammatory drug through its activity as a corticosteroid hormone receptor agonist. Paclitaxel can alter the inflammatory phenotype of macrophages from an immunosuppressive, M2-like phenotype towards a more proinflammatory, M1-like phenotype and improve the efficacy of other anti-cancer therapies. At neutral pH and oxidative conditions, the constructs retain their payloads long enough to carry them into an endosome. Once inside an acidified endosome with potentially reducing conditions and/or appropriate enzyme activity, the drug payload is released. These dexamethasone- and paclitaxel-carrying MADs have low toxicity to human macrophages but a remarkable ability to alter their phenotypes. In the case of the dexamethasone carrying MAD construct (MAD-DEX), MAD-DEX induced macrophages to adopt a more immunosuppressive, M2-like phenotype. The paclitaxel carrying MAD construct (MAD-PAC) induced macrophages to adopt a highly proinflammatory, M1-like phenotype that could not be fully duplicated by unbound (free) paclitaxel.

According to some embodiments, a compound is provided comprising a polymeric carbohydrate backbone, one or more mannose-binding C-type lectin receptor targeting moieties, and a therapeutic agent comprising one or more reactive hydroxyl groups, and coupled to the polymeric carbohydrate backbone via a degradable linker as discussed herein. In further embodiments, the compound comprises a subunit as shown in Formula (I) below:

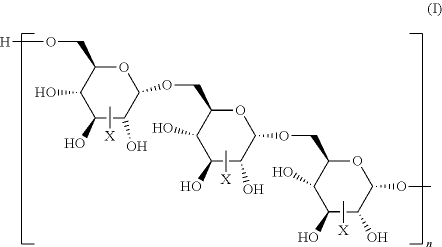

wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

According to some embodiments, at least one X is L₁-A-Z, wherein at least one X is L₂-R, and wherein R comprises a mannose-binding C-type lectin receptor targeting moiety as discussed herein. In some aspects, at least one L₁ comprises —(CH₂)ₚS(CH2)_{q}—NH—, wherein p and q are integers from 0 to 5. In some further aspects, at least one L₂ comprises —(CH₂)ₚS(CH2)_{q}—NH—, wherein p and q are integers from 0 to 5. In some embodiments, the therapeutic agent is conjugated to the degradable linker prior to being coupled to the polymeric carbohydrate backbone.

In some aspects, n is an integer greater than zero. In other aspects, n is an integer greater than 1. In further aspects, n may be an integer between 1 and about 50, between about 5 and about 40, or between about 5 and about 30. As would be understood by those skilled in the art, each subunit of n may be the same or may be different, as the order of each X attached to the polymeric carbohydrate backbone may be the same or different per each subunit of n.

In some embodiments, the degradable linker has the following formula below prior to being conjugated to the therapeutic agent and the polymeric carbohydrate backbone:

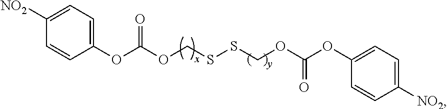

wherein x is an integer between 1-5, and wherein y is an integer between 1-5.

In further embodiments, the degradable linker has the following formula below prior to being conjugated to the therapeutic agent and the polymeric carbohydrate backbone:

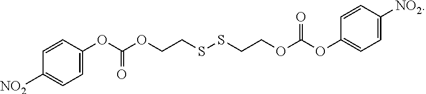 [SMILES: O=C(OCCSSCCOC(=O)Oc1ccc([N+](=O)[O-])cc1)Oc1ccc([N+](=O)[O-])cc1]

In embodiments, A has the following formula:

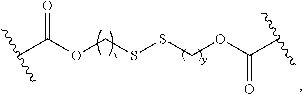

wherein x is an integer between 1-5, and wherein y is an integer between 1-5.

In further embodiments, A has the following formula:

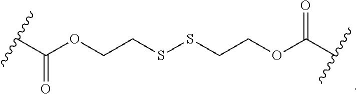 [SMILES: *C(=O)OCCSSCCOC(*)=O]

In some embodiments, a compound comprising a therapeutic agent comprising a reactive hydroxyl group, a degradable linker comprising one or more carbonate and/or disulfide moieties; and a second compound comprising a primary amine, wherein the degradable linker is coupled to the reactive hydroxyl group of the therapeutic agent and coupled to the primary amine of the second compound is provided. In an aspect, the compound comprises a compound according to Formula (II):

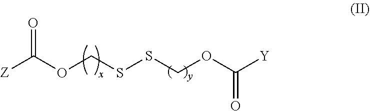

wherein
Z is the therapeutic agent comprising a reactive hydroxyl group;
Y is the second compound comprising a primary amine;
x is an integer between 1-5; and
y is an integer between 1-5.

In certain implementations, Z is a therapeutic agent as discussed herein. In further implementations, Y is a second compound comprising a primary amine group. The compound according to Formula (II) further includes a carbonate moiety and a disulfide moiety. In some aspects, any hydrogen groups within Formula (II) may be substituted with a linear or branched alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) group.

In further embodiments, the therapeutic agents disclosed herein may be attached to the polymeric carbohydrate constructs disclosed herein. In some aspects, the therapeutic agents are modified with a degradable linker comprising a carbonate and/or disulfide linker prior to being attached to the polymeric carbohydrate-based backbone via one of the amine-terminated leashes. In certain embodiments where the therapeutic agent modified with a carbonate/disulfide linker are to be attached to the MAD construct, the polymeric carbohydrate-based backbone already comprises one or more leashes attached to the backbone. Without being limited to any particular theory or mechanism, the drug payload is released upon being cleaved by a reducing agent. Examples of reducing agents may include L-Glutathione (GSH), or other free thiol groups.

According to certain embodiments, the disclosed compounds can include a pharmaceutically acceptable carrier and a compound or a pharmaceutically acceptable salt of the compounds disclosed herein. The disclosed compounds, or pharmaceutically acceptable salts thereof, can also be included in pharmaceutical compositions in combination with one or more other therapeutically active compounds. In certain aspects, the compound is administered in a therapeutically effective amount. In further aspects, the compound is administered in a prophylactically effective amount. In some embodiments, the pharmaceutical compositions are prepared in a form suitable for intravenous, intraperitoneal, or intramuscular injection.

Molecular weights referenced herein, as well as the number and degree of conjugation of receptor substrates, leashes, and therapeutic moieties attached to the polymeric carbohydrate backbone refer to average amounts for a given quantity of carrier molecules, since the synthesis techniques will result in some variability.

### Methods

In living humans and animals, activated macrophages have been observed that have a mixed activated phenotype with features of both M1-like and M2-like phenotypic states. Examples of stimuli that can induce an M1-like phenotype in macrophages include tumor necrosis factor (TNF), interferon gamma (INFy), and toll-like receptor (TLR) agonists such as lipopolysaccharide (LPS). Examples of stimuli that can induce an M2-like phenotype in macrophages include interleukin 4 (IL4), interleukin 13 (IL13), and tumor growth factor beta (TGF8). There are many other hormones, cytokines, chemokines, and environmental factors that can also affect macrophage phenotypes.

Examples of cell surface markers and secreted proteins that frequently have altered levels of expression in M1-like and M2-like activated macrophages are shown in Table 1. In addition, two immune checkpoint receptors, PD-1 and SIRPa, can be expressed on the surface of macrophages. When these immune checkpoint receptors bind to their ligands, PD-L₁ and CD47 respectively, a signal is generated that represses a macrophage's phagocytic activity. An M1-like macrophage would be expected to attack and phagocytize perceived pathogens or tumor cells. However, if the M1-like macrophage was expressing PD-1 and/or SIRPa that had bound to its ligand, phagocytosis would be repressed.

TABLE 1
Examples of Proteins that Increase in Expression in Activated
Macrophages with M1-Like and M2-Like Phenotypes

| | |
|---|---|
| Increased in M1-like | Increased in M2-like |

| Cell Surface | Secreted | Cell Surface | Secreted |
|---|---|---|---|
| MHC II | TNF | CD163 | IL-10 |
| CD80 | IL-1 | CD206 | IIL-13 |
| CD86 | IL-12 | | TGFβ |

Under the influence of M2-like TAMs, the tumor immune microenvironment becomes tumor promoting and immunosuppressive, repressing the anti-tumor activity of other immune cells, such as lymphocytes. In some aspects, the tumor promoting and immunosuppressive activities of M2-like TAMs reduce the efficacies of anti-cancer therapies and, perhaps most notably, of anti-cancer immunotherapies. For these reasons, TAMs are an important therapeutic target for cancer. TAM targeted cancer therapeutic strategies include: (1) killing or ablating TAMs; (2) blocking recruitment of TAMs to tumors; or (3) causing TAMs to switch their phenotypes from M2-like to M1-like. This third strategy is sometimes referred to as TAM repolarization or TAM reeducation. M1-like TAMs will attack tumor cells and stimulate other types of immune cells, such as lymphocytes, to do the same.

In certain embodiments, a method of repolarizing a TAM from an immunosuppressive (M2-like) phenotype to a proinflammatory (M1-like) phenotype is disclosed. In further embodiments, a method of reducing macrophage-mediated inflammation is provided. In certain aspects, polymeric carbohydrate constructs carrying a therapeutic agent, are appended via a degradable linker comprising a carbonate and/or disulfide linkage and linked to the polymeric carbohydrate-based backbone using an amine terminated leash. In certain aspects, at a neutral pH, the constructs retain their payloads (i.e. the therapeutic agents) long enough to carry them to a mannose-binding C-type lectin receptor (such as, for example, CD206). Once the mannose-binding C-type lectin receptor binds to the construct, the receptor/ligand complex is internalized by receptor mediated endocytosis to endosomes that become naturally acidified to a pH of about 4 to 5. Once inside an acidified endosome, the drug payload is released. In some aspects, the polymeric carbohydrate constructs have low toxicity to human macrophages, however, have a remarkable ability to alter their phenotype to become more proinflammatory and anti-tumor.

In certain implementations, the methods comprise administering to a subject in need thereof an effective dose of a compound disclosed herein. In further embodiments, the methods comprise administering a compound comprising a polymeric carbohydrate backbone, one or more mannose-binding C-type lectin receptor targeting moieties, and a therapeutic agent coupled to the polymeric carbohydrate backbone via a carbonate/disulfide linker. In even further embodiments, the compound comprises the subunit as provided in Formula (I) disclosed herein.

In certain aspects, the compound is administered in a therapeutically effective amount. In further aspects, the compound is administered in prophylactically effective amount.

In yet further aspects, the methods further comprise administering the compound intravenously, intraperitoneally, intramuscularly, orally, subcutaneously intraocularly, intra-tumor injection or transdermally or delivered directly to tumor organ by invasive techniques.

In still further aspects, the methods further comprise administering the composition in conjunction with at least one other treatment or therapy. In some aspects, the other treatment or therapy comprises an anti-inflammatory agent. In further aspects, the other treatment or therapy comprises co-administering an anti-cancer agent. In further aspects, the other treatment or therapy is chemotherapy. In certain aspects, the compound is administered alone or in combination with other chemical-based therapeutics or with radiation therapy or thermal therapy or physical therapy or dietary therapy.

According to further embodiments, the at least one other treatment or therapy is an immunotherapy, such as, the administration of an immunomodulatory agent. According to certain implementations, the at least one other treatment or therapy is anti-CTLA4 immunotherapy. In certain implementations, the immunomodulatory agent is an immunostimulator. In some embodiments, the immunomodulatory agent is a glucocorticoid, hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a cytostatic agent, an alkylating agent, nitrogen mustard (cyclophosphamide), nitrosourea, a platinum compound, an antimetabolite, a purine analog, azathioprine, mercaptopurine, mycophenolic acid, a pyrimidine synthesis inhibitor, leflunomide, teriflunomide, a folic acid analog, methotrexate, a cytotoxic antibiotic, dactinomycin, anthracyclines, mitomycin C, bleomycin, mithramycin, an antibody or fusion thereof, anti-thymocyte globulin, anti-lymphocyte globulin, an anti-IL-2 receptor antibody, anti-IL6 antibody, an anti-CD3 antibody, OKT3 (muromonab), otelixizumab, teplizumab, visilizumab, an anti-CD4 antibody, clenoliximab, keliximab, zanolimumab, an anti-CD11a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, ipilimumab, tremelimumab, an anti-eotaxin 1 antibody, bertilimumab, an anti-α4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an immunophilin modulating agent, rapamycin, a calcincurin inhibitor, tacrolimus, ciclosporin (cyclosporin), pimccrolimus, abetimus, gusperimus, ridaforolimus, everolimus, temsirolimus, zotarolimus, a TNF inhibitor, infliximab, adalimumab, certolizumab pegol, golimumab, etanercept, thalidomide, lenalidomide, pentoxifylline, bupropion, curcumin, catechin, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, an opiod, an IMPDH inhibitor, mycophenolic acid, myriocin, fingolimod, an NF-KB inhibitor, raloxifene, drotrecogin alfa, denosumab, an NF-KB signaling cascade inhibitor, disulfiram, olmesartan, dithiocarbamate, a proteasome inhibitor, bortezomib, MG132, Pro1, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C(indole-3-carbinol)/DIM(di-indolmethane) (13C/DIM), Bay 11-7082, luteolin, cell permeable peptide SN-50, IKBa-super repressor overexpression, NFKB decoy oligodeoxynucleotide (ODN), or a derivative or analog of any thereof.

In exemplary implementations, the combined administration of the compound and the at least one treatment or therapy is synergistically effective relative to administration of either alone.

According to certain embodiments, administration of the compounds disclosed herein in conjunction with another therapy or treatment is associated with reduced toxicity compared to administration of the other therapy or treatment alone. In further embodiments, the co-administration of the instantly disclosed compounds and other therapy or treatment produce a synergic effect. In yet further embodiments, the co-administration of the instantly disclosed compounds and provides for lower effective dose of the other therapy or treatment.

The methods provided herein may be practiced in an adjuvant setting. In some embodiments, the method is practiced in a neoadjuvant setting, i.e., the method may be carried out before the primary/definitive therapy. In some embodiments, the method is used to treat an individual who has previously been treated. Any of the methods of treatment provided herein may be used to treat an individual who has not previously been treated. In some embodiments, the method is used as a first line therapy. In some embodiments, the method is used as a second line therapy.

In further embodiments, a method of treating a disease is provided. In some embodiments, the method comprises administering to a subject in need thereof an effective amount of a compound as disclosed herein. In some aspects, the disease is selected from the group consisting of cancer, an autoimmune disease, an inflammatory disorder, Non-Alcoholic Steatohepatitis (NASH), acute respiratory distress syndrome (ARDS), sepsis, coronavirus infection, influenza infection, cytokine storms, and other macrophage involved diseases.

According to other aspects, the subject has been diagnosed with melanoma, breast cancer, lung carcinoma, pancreatic carcinoma, renal carcinoma, ovarian, prostate or cervical carcinoma, glioblastoma, or colorectal carcinoma, cerebrospinal tumor, head and neck cancer, thymoma, mesothelioma, esophageal cancer, stomach cancer, liver cancer, pancreatic cancer, bile duct cancer, bladder cancer, testicular cancer, germ cell tumor, ovarian cancer, uterine cervical cancer, endometrial cancer, lymphoma, acute leukemia, chronic leukemia, multiple myeloma, sarcoma, or any combination thereof.

In certain aspects, the methods further comprises administering the composition as a bolus and/or at regular intervals. In certain aspects, the disclosed method further comprises administering the composition intravenously, intraperitoneally, intramuscularly, orally, subcutaneously, intra-tumorally or transdermally.

According to certain further embodiments, the method further comprises diagnosing the subject with cancer. In further aspects, the subject is diagnosed with cancer prior to administration of the composition. According to still further aspects, the method further comprises evaluating the efficacy of the composition. In yet further aspects, evaluating the efficacy of the composition comprises measuring tumor size prior to administering the composition and measuring tumor size after administering the compound. In even further aspects, evaluating the efficacy of the composition occurs at regular intervals. According to certain aspects, the disclosed method further comprises optionally adjusting at least one aspect of method. In yet further aspects, adjusting at least one aspect of method comprises changing the dose of the composition, the frequency of administration of the composition, or the route of administration of the compound.

According to certain alternative embodiments, the subject has been diagnosed with a disease associated with elevated levels of CD206+macrophages and/or MDSC. Such diseases or conditions include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barré syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenic purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, ocular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleritis, scleroderma, sepsis, serum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome,) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

Further provided are methods of making the compounds as disclosed herein. In certain embodiments, the methods of making the compound according to Formula (I) may comprise the following steps: (a) synthesizing a polymeric carbohydrate backbone having one or more amine terminated leashes attached thereto; (b) synthesizing a degradable linker comprising one or more carbonate and/or disulfide moieties; (c) reacting the degradable linker with a reactive hydroxyl group of the therapeutic agent to form a therapeutic-linker compound; and (d) reacting the therapeutic-linker compound with the one of the one or more amine terminated leashes on the polymeric carbohydrate backbone.

In an aspect, the steps (a) through (d) do not need to occur in the exact same order. In further aspects, additional steps may occur between each of steps (a) through (e). In some embodiments, step (a) may occur before step (b), (c), or (d), or may occur after step (b) or (c).

In further embodiments, the therapeutic agent is conjugated to the degradable linker prior to being coupled to the polymeric carbohydrate backbone. Additional discussion regarding the methods of making the compounds may be found within the non-limiting Examples provided herein.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### Examples

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

Mannosylated amine dextrans (MADs) carrying dexamethasone or paclitaxel payloads (MAD-DEX, or MAD-PAC, respectively) as drug delivery constructs targeted to CD206 expressing cells were synthesized under three parts, that need not be in consecutive order: (1) synthesis of the MAD backbones; (2) reaction of a degradable linker with a reactive hydroxyl group of the dexamethasone or paclitaxel to form a therapeutic-linker compound; and (3) reacting the therapeutic-linker compound with one or more amine terminated leashes on the MAD backbone. The constructs were assessed to determine their ability to release the therapeutic payloads upon being cleaved by a reducing agent, as well as the effect on inflammatory marker expression in human macrophages.

### Example 1: Synthesis of the MAD Delivery Constructs

Synthesis of the MAD backbones: Beginning with a 10 kDa or 3.5 kDa (Mw) dextran, MAD backbones were synthesized as described in U.S. Pat. No. 6,409,990 (which was previously incorporated by reference in its entirety) for mannosylated amine DTPA dextran, with the exception that the conjugation of a chelating agent (i.e. DTPA) was omitted. The resulting construct had a combination of (1) glucose moieties modified by the attachment of amine terminated leashes to which mannose moieties were conjugated, (2) glucose moieties with amine terminated leashes to which mannose moieties were not conjugated (i.e. free amine terminated leashes), and (3) unmodified glucose moieties without either amine terminated leashes or conjugated mannose. The resulting construct had the following structure:

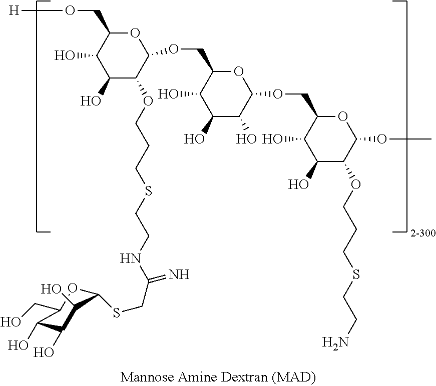

The free amine terminated leashes could be later utilized as points of attachment for drug payloads via degradable linkers. For simplicity in the above structure, the amine terminated leashes are shown attached to the C2 hydroxyl groups of the glucose moieties in the dextran polymer; however, these leashes may be distributed to any of the hydroxyl groups of the MAD backbone.

MAD backbones suitable for drug delivery of therapeutic payloads to CD206 expressing cells can be constructed using initial dextran polymers that range from 1 kDa to greater than 150 kDa in average molecular weight (Mw). Changing the initial Mw of the starting dextran dictates the final size (Mw) of the final drug delivery construct. Drug delivery vehicles of differing Mw vary in their biodistribution when administered to either animal or human subjects. These differences in biodistribution may permit optimizing the drug delivery vehicle for varying pharmacological applications. In addition, the number of amine terminated leashes and/or the number of conjugated mannose moieties can be purposefully changed in theory to range from zero to the number of hydroxyl groups on the glucose moieties of the initial dextran. Changing the numbers of free amines and mannose moieties will also change biodistributions and CD206 receptor affinities, which may permit optimizing the drug delivery vehicle for varying pharmacological applications.

### Example 2: Conjugation of Dexamethasone to MAD Backbones

Various linkers were evaluated to conjugate dexamethasone to a MAD backbone. Successful linkages were determined to be those that allowed for the release of the therapeutic agent/payloads once internalized by endosomes.

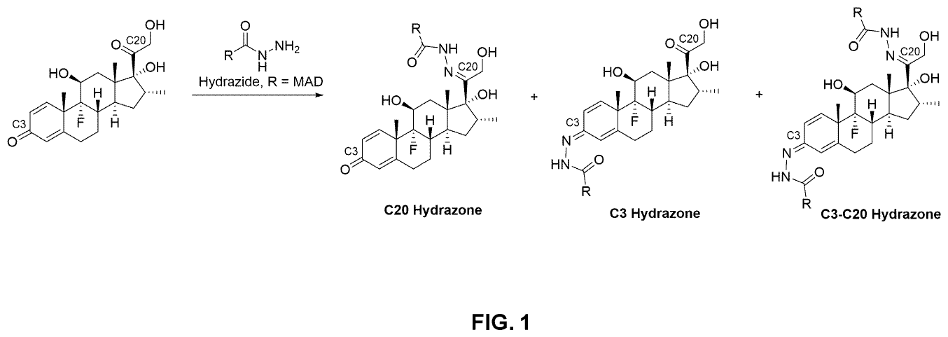

Modification of MAD backbones with dexamethasone via a hydrazone linker: Dexamethasone was conjugated to MAD backbones utilizing a hydrazone formation. The hydrazone formation was completed via condensation of the carbonyl groups on the dexamethasone with hydrazide. The dexamethasone-hydrazone linker compound was then conjugated to MAD backbones to prepare MAD-dexamethasone constructs (also described herein as MAD-dexamethasone conjugates). The reaction methods utilizing a hydrazone linker have been previously disclosed, for example, in U.S. Pat. No. 10,806,803, which is hereby incorporated by reference in its entirety. The reaction between dexamethasone and hydrazide to form various dexamethasone-hydrazone linker compounds is shown in FIG. 1.

While the hydrazide was successfully conjugated to dexamethasone, once the dexamethasone-hydrazone linker compound was conjugated to the MAD backbone, the release profile of dexamethasone from the MAD backbone under aqueous low pH conditions found within endosomal and lysosomal compartments was found to be unacceptable. The reaction of hydrazides with dexamethasone produced a mixture of hydrazones as shown in FIG. 1. The formation at the C3 ketone resulted in a stable thermodynamic product. As such, without being limited to any particular mechanism or theory, it is contemplated that, in some embodiments, the conjugation of the hydrazide at the C3 ketone position of dexamethasone results in a high proportion of the hydrazone formed that is not able to be released from the construct due to the stable thermodynamic product formed, even under highly acidic conditions. Thus, while a hydrazone linker chemistry may be used to attach dexamethasone to a MAD backbone, the resulting construct will not release a significant portion of the dexamethasone payload once internalized to endosomes by a CD206+ cell, for example, and rendering the compound pharmacologically inactive.

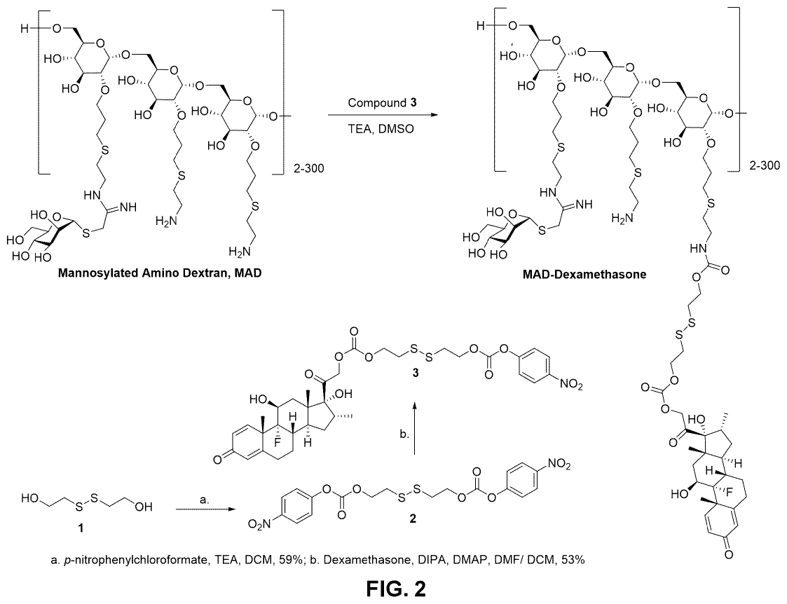

Modification of MAD backbones with a reducing agent sensitive disulfide linker attached to dexamethasone via an ester-linkage: The synthesis and drug release profiles of MAD-dexamethasone constructs were further evaluated with symmetrical disulfides linked to the C21 hydroxyl group of dexamethasone and to the amine terminated leashes of the MAD backbone (FIG. 2). The initial application of this method resulted in the successful synthesis of MAD-drug conjugates via an ester-linkage to dexamethasone (not shown in FIG. 2). Further, the dexamethasone in the ester-linked conjugate was separated from the MAD backbone by disulfide cleavage in a 10 mM aqueous solution of glutathione, and at a pH of 4.65. Without being limited to any particular theory or mechanism, it is contemplated that the dexamethasone payloads linked in this manner were released upon intracellular uptake of the MAD construct where an abundance of free thiols reductively cleaved the disulfide linker. However, the ester linkage remained intact with the dexamethasone and no free dexamethasone could be detected after more than 24 hours.

Modification of MAD backbones with a reducing agent sensitive disulfide linker attached to dexamethasone via a carbonate-linkage: MAD backbones were further evaluated after the symmetrical disulfide/carbonate linker 3 was bonded to dexamethasone as a carbonate as shown in FIG. 2. The dexamethasone and disulfide/carbonate linker 3 compound (which may be further referred to herein as “dexamethasone-carbonate compound”) as shown in FIG. 2 was synthesized as follows: 2,2′-dithiodiethanol (1.0 g, 6.5 mmol) was solubilized in 26 ml of anhydrous dichloromethane (DCM). Triethylamine (3.6 ml, 25.9 mmol) and a solution p-nitrophenylchloroformate in 8.5 ml of DCM (2.87g, 14.3 mmol) were added and the reaction allowed to stir under nitrogen at room temperature overnight.

The solution was concentrated and purified on a silica column using a 0-30% gradient of ethyl acetate in hexane, yielding 1.86 g (59%) of dinitrophenyl carbonate 2 as shown in FIGS. 2. To 500 mg (1.03 mmol) of the dinitrophenyl carbonate 2 in 30 ml of anhydrous dichloromethane under nitrogen is slowly added a 10 ml 1:1 anhydrous N,N-dimethylformamide (DMF) and DCM solution of diisopropylamine (0.27 ml, 1.55 mmol) and 203 mg (0.516 mmol) of dexamethasone. Further, 13 mg (20 mol %, 0.103 mmol) of dimethylaminopyridine in 5 ml of DCM was then added and the solution stirred under nitrogen at room temperature and protected from light. The reaction was monitored by TLC for the disappearance of the starting dexamethasone, diluted with ethyl acetate, extracted with saturated ammonium chloride and brine, dried over sodium sulfate and concentrated. Silica column chromatography using a 10-60% gradient of ethyl acetate in hexane provided 203 mg (53%) of the dexamethasone-carbonate compound as an off-white crystalline solid. Surprisingly, these dexamethasone-carbonate compounds conjugated to a MAD backbone efficiently and resulted in MAD-dexamethasone constructs with the appropriate release profile under reducing conditions (FIG. 2). These beneficial effects are further described within the examples herein.

To prepare a MAD-dexamethasone construct on a 10 kDa MAD backbone (25 mannose, 12 available amines), 100 mg of MAD (5.0 μmol) was dissolved in 4 ml of anhydrous dimethylsulfoxide (DMSO) using brief sonication and mild heating. The solution was cooled to ambient temperature and 6 equivalents (4.2 μl, 30 μmol) of triethylamine (TEA) followed by 6 equivalents (22.3 mg, 30 μmol) of solid dexamethasone-carbonate compound 3 was added. At 12 hours, a sample of the reaction solution was tested and compared to a time zero sample for loss of amine-content which was equivalent to the addition of 3.8 dexamethasone-carbonate compounds bound as carbamate to the MAD backbone. The reaction solution was cooled on an ice bath and diluted slowly with 18 ml of purified water. The solution was concentrated on a centrifugal spin-filter containing a 10 kDa MWCO (molecular weight cut-off) membrane, followed by diluting and concentrating with two volumes of purified water, two volumes of 50% ethanol and two volumes of purified water. The retentate containing the product was removed from the membrane with purified water, then frozen and lyophilized, which resulted in 60 mg of MAD-dexamethasone conjugate as a bright yellow foam. The total dexamethasone in the conjugate was determined by UV at 239 nm, and free unbound dexamethasone (0.36% by weight) was determined by standard curve and HPLC analysis. Bound dexamethasone was determined to be 6.91% by weight, corresponding to an average of 3.92 dexamethasone moieties per MAD on the 10 kDa backbone.

To prepare a MAD-dexamethasone construct on a 3.5 kDa MAD backbone (11 mannose, 6 available amines), 100 mg of MAD (12.4 μmol) was dissolved in 4 ml of anhydrous DMSO and 3 equivalents (5.2 μl, 37 μmol) of TEA followed by 3 equivalents (27.4 mg, 37 μmol) of solid dexamethasone-carbonate 3. At 12 hours, the reaction solution was cooled on an ice bath and diluted slowly with 18 ml of purified water. The solution was concentrated on a centrifugal spin-filter containing a 3 kDa MWCO membrane, followed by diluting and concentrating with five volumes of purified water. The retentate containing the product was removed from the membrane with purified water, then frozen and lyophilized, which resulted in 98 mg of the MAD-dexamethasone conjugate as a bright yellow foam. The total dexamethasone in the conjugate was determined by UV at 239 nm, and free unbound dexamethasone (0.74% by weight) by standard curve and HPLC analysis. Bound dexamethasone was determined to be 7.77% by weight, corresponding to an average of 1.88 dexamethasone moieties per MAD on the 3.5 kDa backbone.

### Example 3: Release of Dexamethasone from MAD-Dexamethasone Conjugate (10 mM GSH, 37° C.)

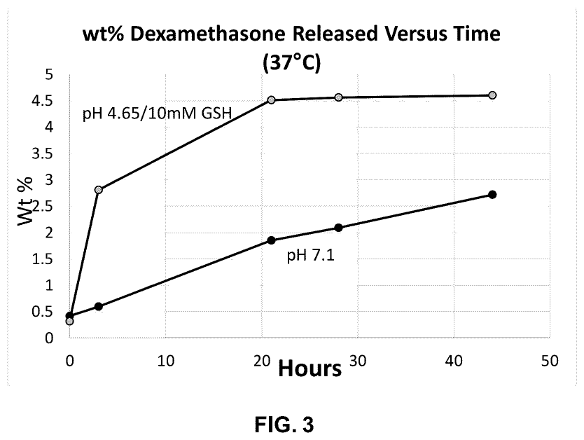

After the synthesis of MAD-dexamethasone on a 10 kDa dextran backbone was complete, samples were evaluated over time for the release of dexamethasone. Two 1 mg/ml solutions of construct were prepared: one in 0.1 M PBS buffer at pH 7.1, and the other in 0.1 M sodium acetate buffer at a pH of 4.65 and 10 mM of glutathione. Each sample was sealed and shaken at 37° C. and monitored by HPLC from time zero to 48 hours. The amount of dexamethasone released from the construct was determined from a dexamethasone standard curve. Dexamethasone released under the pH 4.65 and reducing conditions increased from 3 to 20 hours (FIG. 3), and at 21 hours, 4.5% by weight (2.5 drug moieties per dextran chain or 71% of bound drug) of free dexamethasone was detected by HPLC. By comparison, about 1 dexamethasone was released at pH 7.1 under non-reducing conditions at 21 hours, which is well outside the timeframe that MAD constructs circulate in the blood. This is an important feature of the MAD constructs disclosed herein as it permits gradual and continuous release of the drug payload into CD206 expressing cells which would not have been possible if the drug were not delivered on a MAD backbone.

### Example 4: MAD-Dexamethasone Conjugates—Evaluations in Human Macrophage Cell Culture Assays

The MAD-dexamethasone conjugates (MAD-DEX) were evaluated in a human macrophage cell culture assay. In this assay, human peripheral blood monocytes (hPBMCs) were incubated for five days in RPMI+10% FBS+1x Penicillin/Streptomycin/L-glutamine+5Ong/mlGM-CSF (complete medium) at a concentration of 500,000 monocytes per well in 48 well tissue culture plates. During this five-day incubation, the monocytes differentiated into macrophages. GM-CSF induces the macrophages to adopt an activated phenotype that is intermediate between the extremes of M1 or M2. After the five-day incubation, the medium was removed and replaced with complete medium supplemented with various concentrations of MAD-DEX or unbound dexamethasone (free dexamethasone). Saline and Vehicle (MAD without a drug payload, 23.3 μg/ml) were added as alternative supplements to other cultures as negative controls. The macrophage cell cultures were incubated with the supplemented complete medium for 24 hours after which it was removed and replaced with fresh complete medium. The macrophage cultures were then incubated for an additional three days. The three-day post treatment incubation was performed to permit assessment of durable changes in macrophage phenotypes.

After the additional three-day incubation in fresh complete medium, the cells were harvested and evaluated by flow cytometry for viability (DAPI) or expression of macrophage surface markers. These markers are representative of markers that are frequently considered as indicative of either M1-like or M2-like phenotypes or are known immune checkpoint receptors. The evaluated cell surface markers included CD206, CD163, CD80, CD86, MHC1, MHC2, SIRPa, and PD-1 using antibodies specific for each marker. Although the amount observed of each surface marker varied considerably between markers and between treatment groups, nearly all live cells expressed detectable amounts of all markers. The marker with the lowest expression in saline and vehicle treated controls was PD-1. The outputs of the flow cytometry assays were mean fluorescent intensities (MFI). Macrophages differentiated from monocytes collected from three separate donors were evaluated for all treatments. For each donor's macrophages, all experiments were run in triplicate.

Results: In all replicates of this example, the large majority of macrophages treated with the saline control survived to the end of the experiment (9 days). For cell viability and for all surface markers evaluated, the results for the drug free vehicle treated control were not statistically different from those observed for the saline (no drug) control, indicating that the drug free vehicle did not have pharmacological activity observed in this study. Furthermore, neither MAD-DEX nor free dexamethasone decreased cell viability at any tested concentration.

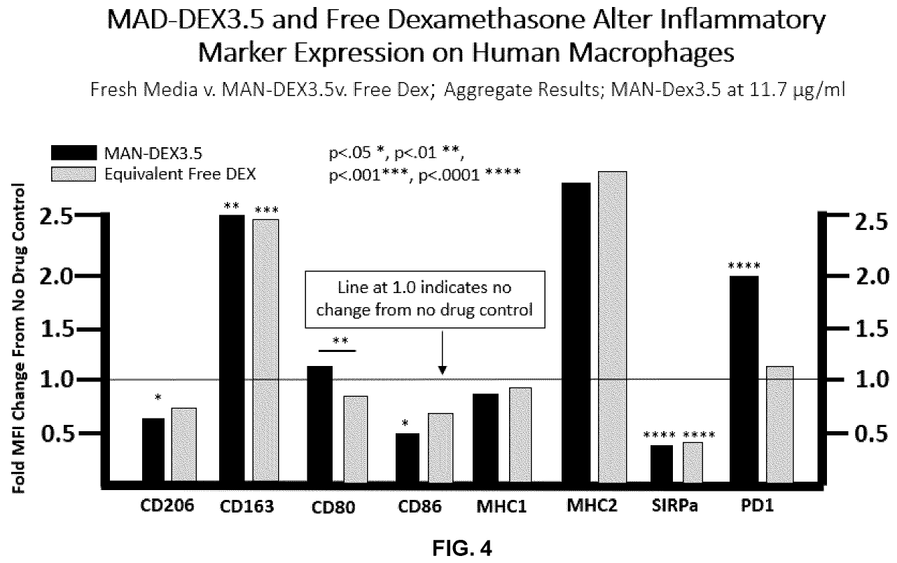

FIG. 4 shows an aggregate representation of how the various treatments with either MAD-DEX3.5 (MAD-DEX built on a 3.5 kDa dextran backbone) or free dexamethasone affected the level of expression of the eight evaluated surface markers. The values shown are the average fold change in expression (MFI) compared to the MFI values observed in the saline controls. The statistical significance of these changes in expressions was measured by Z tests.

The data shown in FIG. 4 shows the fold changes in expression of the indicated surface markers treated with either 11.7 μg/ml of MAD-DEX3.5 or an equal molar amount of free dexamethasone not attached to the MAD vehicle. A fold change of 1.0 indicated that there was no change in expression, i.e., the average level of expression (MFI) observed in the treated cells was the same as what was observed in the saline controls. A value of 2.0 means that the average MFI of the treated cells was twice that of the MFI observed in the saline treated controls. Conversely, a value of 0.5 means that the MFI observed in the treated cells was half that observed in the saline controls.

Several important observations can be made from the data represented in FIG. 4. First, MAD-DEX3.5 and free dexamethasone (at the same concentration as the amount of dexamethasone that had been loaded onto the MAD-DEX3.5 backbone), induced similar changes in macrophage phenotype. Both MAD-DEX3.5 and free dexamethasone significantly increased the expression of CD163, which is typically viewed as a marker for M2-like phenotypes. CD163 may be increased in expression by treatment with corticosteroids such as dexamethasone. Although not as statistically significant, both also reduced expression of CD86, which is typically viewed as a marker for M1-like phenotypes. Interestingly, both also significantly decreased expression of SIRPa and induced a non-significant increase in expression of MHC2. Further, while expression of PD-1 was increased by MAD-DEX3.5, the very low levels of PD-1 expression in macrophages treated with the saline control suggests that the modest but significant increase in PD-1 expression induced by MAD-DEX3.5 may not be phenotypically significant.

The results shown in FIG. 4 are significant for a few reasons. Free dexamethasone is known to be highly anti-inflammatory and the observation that MAD-DEX3.5 induces the same phenotypic changes as free dexamethasone suggests that MAD-DEX3.5 is also highly anti-inflammatory. Further, MAD-DEX3.5 was found to be equally potent to an equivalent molar dose of free dexamethasone. Dexamethasone is lipophilic and freely crosses the cell membrane. MAD constructs are highly hydrophilic and may enter cells if transported across the cell membrane by CD206. Thus, the observation that MAD-DEX3.5 efficiently alters the phenotype of macrophages suggests that it is being transported into macrophages by CD206. As a result, cells not expressing CD206 would not be expected to receive dexamethasone delivered by MAD-DEX3.5.

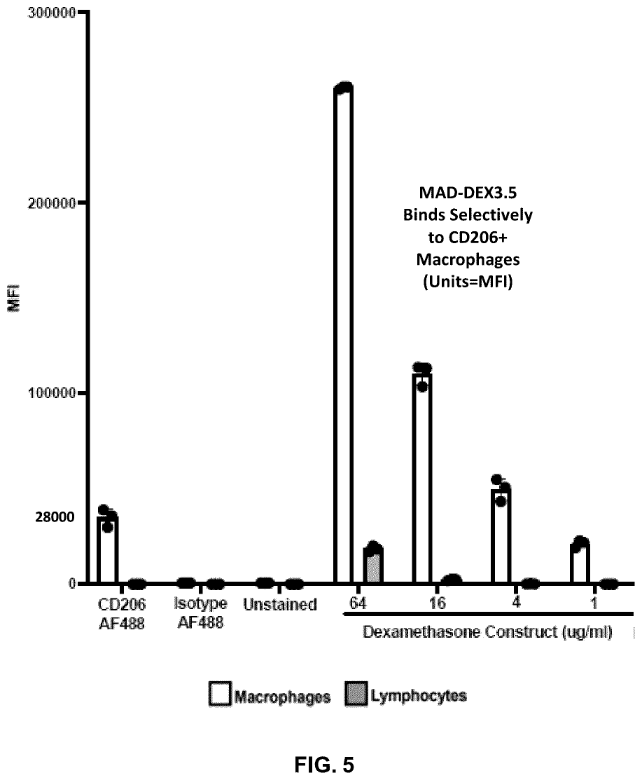

To test the hypothesis that MAD-DEX constructs, such as MAD-DEX3.5, deliver dexamethasone selectively to CD206 expressing cells, such as CD206 positive macrophages, MAD-DEX3.5 was labeled with Alexa Fluor 488 (AF488), a fluorescent marker that permits the localization of MAD-DEX3.5 to various cell populations to be evaluated by flow cytometry. CD206 positive macrophages differentiated for peripheral blood monocytes in complete media as described previously and fresh peripheral blood mononuclear cells (predominately lymphocytes) that do not express CD206 were exposed to increasing concentrations of AF488-MAD-DEX3.5. Cells were then washed and evaluated by flow cytometry for AF488 mediated fluorescence. Control groups comprised cells exposed to an AF488 labeled anti-CD206 monoclonal antibody or antibody isotope controls, and untreated cells. Results are shown in FIG. 5. FIG. 5 demonstrates that AF488-MAD-DEX3.5 binds selectively to macrophages and that only macrophages bound the anti-CD206 antibody, indicating that MAD-DEX3.5, and by inference, all other MAD-DEX constructs, delivers dexamethasone preferentially to CD206 expressing cells. This finding is significant for purposes of utilizing MAD-DEX for reducing macrophage-mediated inflammation while reducing off-target side effects. The targeted delivery of dexamethasone to CD206 expressing cells on MAD constructs provide for clinical utility for treating illnesses such as NASH, ARDS, coronavirus infection, sepsis, cytokine storms, and macrophage involved autoimmune diseases, such as, but not limited to, rheumatoid arthritis.

### Example 5: Conjugation of Paclitaxel to MAD Backbones

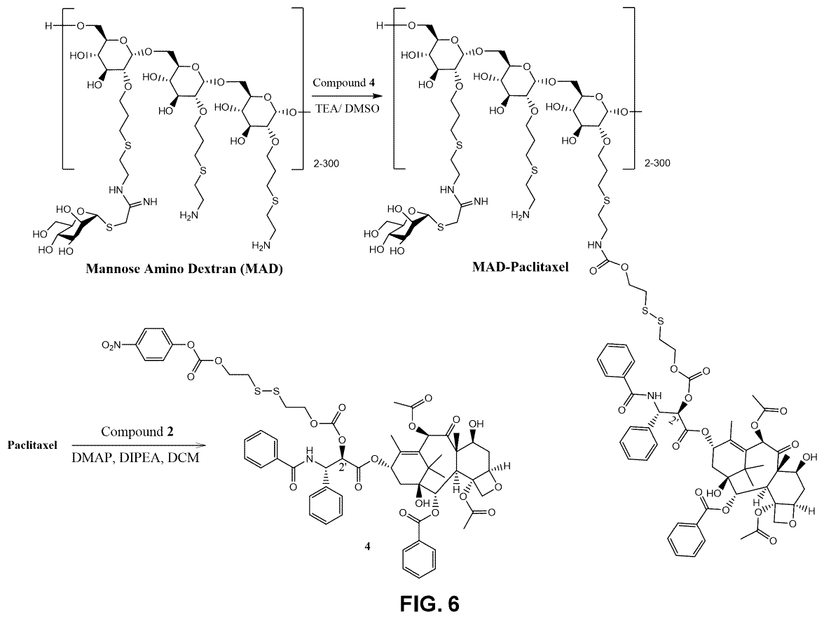

Modification of MAD backbones with a reducing agent sensitive disulfide linker attached to paclitaxel via a carbonate-linkage: The chemical pathways described within Example 2 for a dexamethasone payload on MAD backbones were further evaluated with a different drug payload. As may be contemplated by those skilled in the art, the methods discussed herein may then be adapted to additional drug products bearing a freely accessible and reactive hydroxyl group, and with a wide variety of polymeric backbones. To demonstrate this chemical utility, paclitaxel was modified with the same dinitrophenyl carbonate 2 used for the dexamethasone and disulfide/carbonate linker compound 3 (FIG. 2) to prepare the paclitaxel and disulfide/carbonate linker compound 4 (which may be further referred to herein as “paclitaxel-carbonate compound”) as shown in FIG. 6. In a similar process as described in Example 2, paclitaxel was then conjugated to 3.5 kDa and 10 kDa MAD backbones providing paclitaxel-MAD constructs with desirable release of paclitaxel under reducing conditions.

The paclitaxel-carbonate compound 4 was prepared as follows: 352 mg (0.727 mmol) of dinitrophenyl carbonate 2 was weighed into a vial with a stir bar and dissolved in 18.6 ml of anhydrous DCM under nitrogen. To this clear solution was added 620 mg (0.727 mmol) of paclitaxel (Accela SY016928) followed by 381 μL (2.18 mmol) of diisopropylethylamine (DIPEA) and 20 mol % (17.8 mg, mmol) of DMAP. The reaction solution immediately turned yellow with the addition of base. The vial was protected from light and stirred at ambient temperature for 12 hours. The reaction completion was confirmed by TLC with consumption of starting paclitaxel. The reaction solution was diluted with DCM, extracted with saturated ammonium chloride and brine, dried over anhydrous sodium carbonate, and concentrated. Purification by silica gel chromatography using a 20-80% gradient of ethyl acetate in hexane provided 391 mg of pure paclitaxel-carbonate compound 4.

To prepare the MAD-paclitaxel conjugate (MAD-PAC) on a 10 kDa MAD backbone (18 mannose, available amines), 100 mg of MAD (5.38 μmol) was dissolved in 4 ml of anhydrous DMSO using brief sonication and mild heating. The solution was cooled to ambient temperature and 6 equivalents (4.5 μl, 32 μmol) of TEA followed by 6 equivalents (39 mg, 32 μmol) of solid paclitaxel-carbonate compound 4 was added, whereupon the solution turned bright yellow. After stirring overnight, the reaction was cooled on an ice bath and diluted slowly with purified water. The solution was concentrated on a centrifugal spin-filter followed by diluting and concentrating with three volumes of purified water. The retentate containing the product was removed from the membrane with purified water, then frozen and lyophilized, which resulted in 90 mg of MAD-paclitaxel conjugate as a yellow foam. The free unbound paclitaxel (0.94% by weight) in the conjugate was determined by standard curve and HPLC analysis. Bound paclitaxel was determined using the same HPLC method under releasing conditions (37° C. and 50 mM TCEP at 4 hours) and found to be 12.6% by weight, corresponding to an average of 3.53 paclitaxel moieties per MAD on the 10 kDa backbone.

To prepare the MAD-paclitaxel conjugate on a 3.5 kDa MAD backbone (11 mannose, 6 available amines), 600 mg of MAD (74.2 μmol) was dissolved in 24 ml of anhydrous DMSO using brief sonication and mild heating. The solution was cooled to ambient temperature and 4 equivalents (41 μl, 0.30 mmol) of TEA followed by 4 equivalents (356 mg, 0.30 mmol) of solid paclitaxel-carbonate compound 4 was added, whereupon the solution turned bright yellow. After stirring overnight, the reaction was cooled on an ice bath and diluted slowly with 125 ml of purified water. The solution was transferred with the aid of additional purified water to a stirred-cell fitted with a 3 kDa MWCO membrane and concentrated from 250 ml to approximately 10 ml followed by diluting and concentrating with three additional volumes of purified water. The retentate containing the product was removed from the membrane with purified water, filtered through a 0.2 μm vacuum filter, then frozen and lyophilized, resulting in 525 mg of MAD-paclitaxel conjugate as a yellow foam. The free unbound paclitaxel (3.16% by weight) in the conjugate was determined by standard curve and HPLC analysis. Bound paclitaxel was determined using the same HPLC method under releasing conditions (37° C. and 50 mM TCEP at 22 hours) and found to be 16% by weight, corresponding to an average of 2.0 paclitaxel moieties per MAD on the 3.5 kDa backbone.

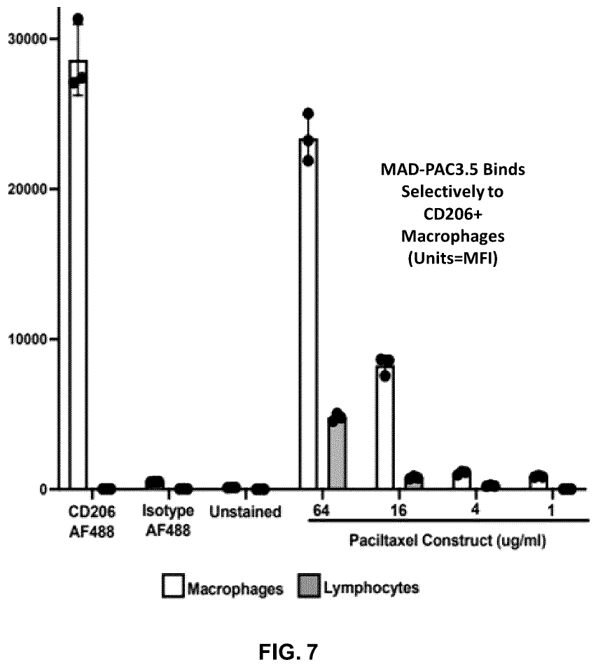

Similar to the experiment described evaluating AF488-MAD-DEX3.5, MAD-PAC3.5 conjugates (built on a 3.5 kDa dextran backbone) were labelled with AF488 to create AF488-MAD-PAC3.5 and evaluated for their ability to selectively bind to macrophages expressing CD206 but not to peripheral blood lymphocytes, which do not express CD206 (See FIG. 7). Similar to the results observed for AF488-MAD-DEX3.5, AF488-MAD-PAC3.5 bound preferentially to macrophages and only macrophages expressed CD206 in this assay. Thus, MAD-PAC3.5, and similar MAD-PAC constructs built on dextran backbones of different molecular weights, will deliver paclitaxel preferentially to CD206 expressing cells such as tumor promoting TAMs while avoiding or limiting off target exposure and toxicities to cells that do not express CD206.

### Example 6: MAD-Paclitaxel Conjugates—Evaluations in Human Macrophage Cell Culture Assays

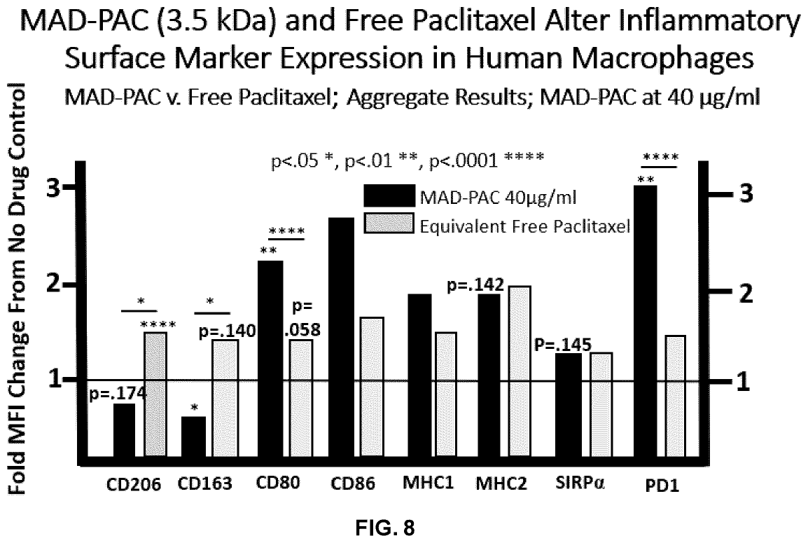

The MAD-PAC3.5 conjugates were further evaluated in human macrophage culture assays similar to the analysis completed in Example 4 for MAD-DEX conjugates. Unlike the previous example of MAD-DEX3.5 and free dexamethasone, MAD-PAC3.5 altered the surface marker expression pattern differently than what was observed for an equal molar equivalent concentration of free paclitaxel as shown in FIG. 8. MAD-PAC3.5 decreased expression of CD206 and CD163 while free paclitaxel modestly increased expression of these markers. MAD-PAC3.5 also increased expression of CD80 and CD86. The decrease in CD206 and CD163 expression coupled with the increase in expression of CD80 and CD86 indicated that MAD-PAC3.5 strongly shifted the immune status of the macrophages towards a more M1-like, proinflammatory phenotype. Free paclitaxel also increased the expression of CD80 and CD86, however, these changes were more modest than observed for MAD-PACX3.5. Importantly, the changes in surface marker expression induced by MAD-PAC3.5 and free paclitaxel were significantly different from one other. Surprisingly, the changes in surface marker expression observed for MAD-PAC3.5 could not be duplicated by free paclitaxel at any drug concentration. MHC1, MHC2, and SIRPa levels of expression were not significantly altered by either drug agent. PD1 was significantly increased by MAD-PAC3.5 but not by free paclitaxel.

These results demonstrate that the targeted delivery of paclitaxel to CD206 expressing cells on MAD constructs could possibly provide clinical utility for treating cancer by altering the phenotype of M2-like, CD206 expressing TAMs to be more M1-like.

### Example 7: MAD-PAC3.5 Reduces Tumor Growth in Combination with Anti-CTLA4 Immunotherapy

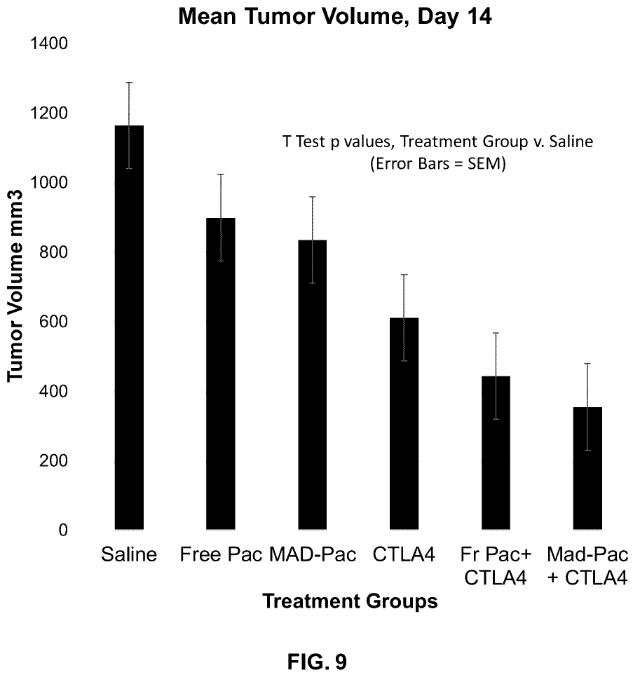

The hypothesis that MAD-PAC3.5 could provide clinical utility for treating cancer was evaluated using the CT26/Balb-c syngeneic mouse tumor model in an experiment in which tumor bearing mice were treated with MAD-PAC3.5 alone or in combination with anti-CTLA4 immunotherapy. Mice were implanted with CT26 cells that were permitted to grow and become established until they had attained tumor volumes of 80-100 mm 3. Tumor bearing mice (n=10/treatment group) were administered test articles by intravenous injections on 4 days (treatment days, 1, 4, 7, and 11). Treatments were saline, MAD without payload (127 μg/injection), MAD-PAC3.5 alone (127 μg/injection), a molar equivalent dose of free paclitaxel, anti-CTLA4 antibody treatment alone, combination therapy with free paclitaxel (equal molar dose) plus anti-CTLA4 therapy, and combination MAD-PAC3.5 (127 μg/injection) plus anti-CTLA4 therapy. Tumor volumes were measured and recorded at regular intervals. Results for mean tumor volumes per treatment group observed on treatment day 14 are shown in FIG. 9. The MAD without payload treatment group returned results that were not different from the saline treatment (not shown). Both free paclitaxel and MAD-PAC3.5 treatments yielded modestly decreased mean tumor volumes, but these results did not reach statistical significance. In this experiment, treatment with anti-CTLA4 antibodies alone reduced mean day 14 tumor volumes by nearly half, which did reach statistical significance (p=0.01). Combination therapy with anti-CTLA4 antibodies and either free paclitaxel or MAD-PAC3.5 further reduced mean day 14 tumor volumes (p=0.005 and p=0.0008 respectively compared to the saline control group). The treatment group that received the combination of anti-CTLA4 and MAD-PAC3.5 had the lowest mean day 14 tumor volumes, which was 76% less than the saline control.

This experiment, in combination with the results of the selective binding assay, indicate that the enhanced tumor control observed in the mice treated with combination of anti-CTLA4 and MAD-PAC3.5 can be achieved without exposing cells and tissues that do not express CD206 to the potential off target cytotoxic effects of paclitaxel, thus increasing the safety of a combination therapy consisting of MAD-PAC3.5 (or other MAD-PAC construct) plus an immunotherapy, such as anti-CTLA4. It is anticipated that similar results may be observed when MAD-PAC3.5 (or similar MAD-PAC construct) is combined with anti-PD1 or anti-PD-L₁ immunotherapy. Similarly, since MAD-PAC3.5 was observed to shift macrophage phenotypes towards a more M1-like immune status, it is anticipated that MAD-PAC constructs may enhance the effectiveness of other anti-cancer therapies such as radiation-based therapies and conventional chemotherapies.

The disclosures being thus described, it will be obvious that the same may be varied in many ways. Such variations are not to be regarded as a departure from the spirit and scope of the disclosures and all such modifications are intended to be included within the scope of the following claims.

What is claimed is:

1. A compound comprising:
a polymeric carbohydrate backbone;
one or more mannose-binding C-type lectin receptor targeting moieties; and
a therapeutic agent comprising one or more reactive hydroxyl groups, and coupled to the polymeric carbohydrate backbone via a degradable linker.

2. The compound of claim 1, wherein the compound comprises a subunit as shown in Formula (I): 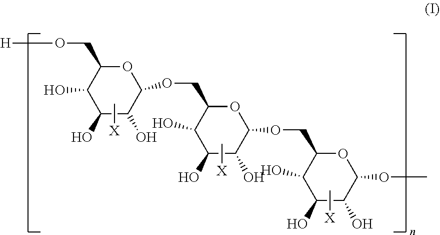
wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

3. The compound of claim 2, wherein at least one X is L₁-A-Z, wherein at least one X is L₂-R, wherein R comprises the mannose-binding C-type lectin receptor targeting moiety, and wherein the degradable linker comprises one or more carbonate and/or disulfide moieties.

4. The compound of claim 1, wherein the mannose-binding C-type lectin receptor targeting moiety comprises a mannosyl coupling reagent, mannose, high-mannose glycans or mannose oligosaccharides, fucose, n-acetylglucosamine, peptides, galactose, or a combination thereof.

5. The compound of claim 2, wherein at least one L₁ and/or at least one L₂ comprises —(CH₂)ₚS(CH2)_{q}—NH—, wherein p and q are integers from 0 to 5.

6. The compound of claim 2, wherein the degradable linker has the following formula below prior to being conjugated to the therapeutic agent and the polymeric carbohydrate backbone: 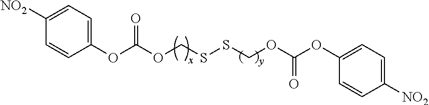
wherein x is an integer between 1-5, and wherein y is an integer between 1-5.

7. The compound of claim 2, wherein A has the following formula: 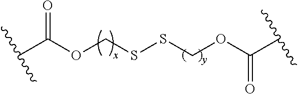
wherein x is an integer between 1-5, and wherein y is an integer between 1-5.

8. The compound of claim 1, wherein the therapeutic agent comprises a corticosteroid, a cortisol, a glucocorticoid-receptor ligand, a chemotherapeutic agent, a toll-like receptor agonist or antagonist, or a combination thereof.

9. The compound of claim 8, wherein the therapeutic agent comprises dexamethasone or paclitaxel.

10. A pharmaceutical composition comprising:
the compound according to claim 1; and
a pharmaceutically effective carrier.

11. The composition of claim 10, wherein the compound comprises a subunit as shown in Formula (I): 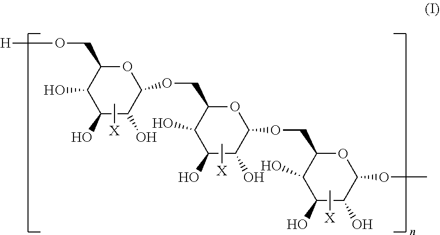
wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

12. A compound comprising:
a therapeutic agent comprising a reactive hydroxyl group;
a degradable linker comprising one or more carbonate and/or disulfide moieties; and
a second compound comprising a primary amine,
wherein the degradable linker is coupled to the reactive hydroxyl group of the therapeutic agent and coupled to the primary amine of the second compound.

13. The compound of claim 12, wherein compound has the following formula (II): 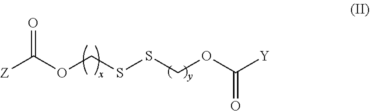
wherein
Z is the therapeutic agent comprising a reactive hydroxyl group;
Y is the second compound comprising a primary amine;
x is an integer between 1-5; and
y is an integer between 1-5.

14. The compound of claim 12, wherein the therapeutic agent comprises a corticosteroid, cortisol, a glucocorticoid-receptor ligand, a chemotherapeutic agent, a toll-like receptor agonist or antagonist, or a combination thereof.

15. A method of making the compound according to claim 2, the method comprising:
(a) synthesizing a polymeric carbohydrate backbone having one or more amine terminated leashes attached thereto;
(b) synthesizing a degradable linker comprising one or more carbonate and/or disulfide moieties;
(c) reacting the degradable linker with a reactive hydroxyl group of the therapeutic agent to form a therapeutic-linker compound; and
(d) reacting the therapeutic-linker compound with the one of the one or more amine terminated leashes on the polymeric carbohydrate backbone.

16. The method of claim 15, wherein the therapeutic agent is conjugated to the degradable linker prior to being coupled to the polymeric carbohydrate backbone.

17. The method of claim 15, wherein at least one X is L₁-A-Z, wherein at least one X is L₂-R, wherein R comprises the mannose-binding C-type lectin receptor targeting moiety, and wherein the therapeutic agent comprises a corticosteroid, cortisol, a glucocorticoid-receptor ligand, a chemotherapeutic agent, a toll-like receptor agonist or antagonist, or a combination thereof.

18. The method of claim 15, wherein the degradable linker has the following formula below prior to being conjugated to the therapeutic agent and the polymeric carbohydrate backbone: 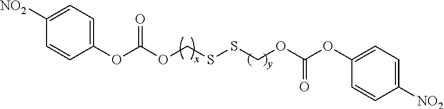
wherein x is an integer between 1-5, and wherein y is an integer between 1-5.

19. The method of claim 15, wherein A has the following formula: 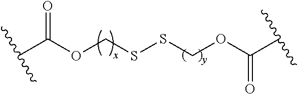
wherein x is an integer between 1-5, and wherein y is an integer between 1-5.

20. A method of repolarizing a tumor associated macrophage (TAM) from an immunosuppressive (M2-like) phenotype to a proinflammatory (M1-like) phenotype, comprising:
administering to a subject in need thereof an effective dose of the compound according to claim 1, comprising a polymeric carbohydrate backbone, one or more mannose-binding C-type lectin receptor targeting moieties, and a therapeutic agent comprising one or more reactive hydroxyl groups, and coupled to the polymeric carbohydrate backbone via a degradable linker.

21. The method of claim 20, wherein the compound comprises a subunit as shown in Formula (I): 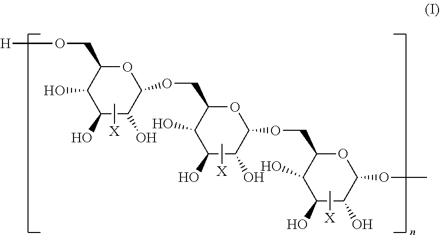
wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

22. The method of claim 20, wherein the compound is administered in conjunction with at least one other therapy or treatment and, wherein the at least one other treatment or therapy comprises chemotherapy, radiation therapy, or immunotherapy.

23. The method of claim 20, wherein the therapeutic agent comprises paclitaxel, and wherein the subject in need thereof is suffering from cancer.

24. The method of claim 20, wherein the therapeutic agent is released from the polymeric carbohydrate backbone in the presence of a reducing agent.

25. A method of reducing macrophage-mediated inflammation, comprising:
administering to a subject in need thereof an effective dose of the compound according to claim 1, comprising a polymeric carbohydrate backbone, one or more mannose-binding C-type lectin receptor targeting moieties, and a therapeutic agent comprising one or more reactive hydroxyl groups, and coupled to the polymeric carbohydrate backbone via a degradable linker.

26. The method of claim 25, wherein the compound comprises a subunit as shown in Formula (I): 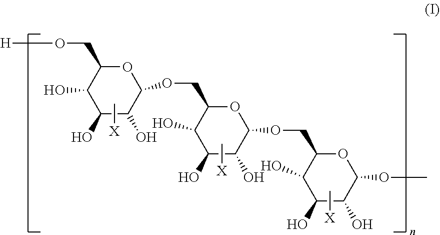
wherein
each X is independently H, L₁-A-Z, or L₂-R, wherein each X is bound to an OH group;
each of L₁ and L₂ are independently amine terminated leashes;
each A independently comprises a degradable linker comprising one or more carbonate and/or disulfide moieties;
each Z independently comprises a therapeutic agent comprising one or more reactive hydroxyl groups;
each R independently comprises the mannose-binding C-type lectin receptor targeting moiety or H; and
n is an integer greater than zero, wherein each unit of n may be the same or different.

27. The method of claim 25, wherein the therapeutic agent comprises dexamethasone, and wherein the therapeutic agent is released from the polymeric carbohydrate backbone in the presence of a reducing agent.

28. The method of claim 25, wherein the subject in need thereof is suffering from Non-Alcoholic Steatohepatitis (NASH), acute respiratory distress syndrome (ARDS), sepsis, coronavirus infection, influenza infection, cytokine storms, other macrophage involved diseases, or a combination thereof.

29. A method of treating a disease, comprising:
administering to a subject in need thereof an effective amount of the compound according to claim 1,
wherein the disease is selected from the group consisting of cancer, an autoimmune disease, an inflammatory disorder, Non-Alcoholic Steatohepatitis (NASH), acute respiratory distress syndrome (ARDS), sepsis, coronavirus infection, influenza infection, cytokine storms, and other macrophage involved diseases.

30. The method of claim 29, wherein the therapeutic agent comprises a corticosteroid, cortisol, a glucocorticoid-receptor ligand, a chemotherapeutic agent, a toll-like receptor agonist or antagonist, or a combination thereof, and wherein the compound is optionally administered in conjunction with at least one other therapy or treatment comprising chemotherapy, radiation therapy, or immunotherapy.
